(19) United States
(12) Patent Application Publication
(10) Pub. No.: US 2024/0425828 A1
(43) Pub. Date: Dec. 26, 2024

(54) TARGETED RETROVIRAL INTEGRATION FOR TREATMENT OF GENETIC DISORDERS

(71) Applicant: Ohio State Innovation Foundation, Columbus, OH (US)

(72) Inventor: Kristine YODER, Hilliard, OH (US)

(21) Appl. No.: 18/274,075

(22) PCT Filed: Jan. 26, 2022
(86) PCT No.: PCT/US2022/013838

§ 371 Date: Jul. 25, 2023

Related U.S. Application Data
(60) Provisional application No. 63/141,677, filed on Jan. 26, 2021.

(51) Int. Cl. C12N 9/12; A61K 48/00; C07K 14/005; C12N 15/86; C12N 15/90

(52) U.S. Cl. CPC C12N 9/1276; A61K 48/005; C07K 14/005; C12N 15/86; C12N 15/907; C07K 2319/81; C12N 2740/17022; C12N 2740/17043; C12Y 207/07049

(57) ABSTRACT

Disclosed herein are methods and compositions related to modified prototype foamy virus (PFV) and its uses to treat diseases and disorders. Also disclosed are methods of making said PFVs.

### I. CROSS REFERENCE TO RELATED APPLICATIONS

This application claims the priority benefit of U.S. Provisional Application No. 63/141,677, filed Jan. 26, 2021, which is expressly incorporated herein by reference in its entirety.

### II. BACKGROUND

Many genetic disorders, such as cystic fibrosis (CF) or severe combined immunodeficiency (SCID), are due to the absence of a wild type gene. Gene therapy delivery of the wild type gene can be a cure to these diseases. Retroviruses do not induce a strong immune response, which allows for repeated delivery of the gene therapy vector if required. Retroviruses also stably integrate the wild type gene into the host genome allowing for stable long-term expression.

Gene therapy vectors derived from the retrovirus murine leukemia virus (MLV) were used to treat SCID in Europe in the 2000s. The MLV vectors delivered a wild type IL-2Rgamma gene to hematopoietic stem cells ex vivo. 11 patients were cured of their life-threatening disease. An additional 6 patients were cured of SCID, but developed leukemia within years of treatment. These tumors were due to the integration of the MLV vector in the promoter of known oncogenes resulting in dysregulation and oncogenesis. In hindsight, it might not be surprising that a virus known to cause leukemia in mice would also generate vector particles leading to leukemia in humans.

Directed integration to specific regions of a host genome can completely avert integration-associated side effects, such as cancer.

Targeted retroviral integration has been a goal in the field of retrovirology since the early 1990s. However, the approaches have largely been brute force attempts at targeting. They have all been unsuccessful for a variety of reasons. In some cases, the retroviral integrase is directed to integrate at a genomic locus by a host co-factor. For example, HIV-1 integration is directed to the body of actively transcribed genes by host protein LEDGF/p75. It will always be impossible to subvert the influence of LEDGF/p75. Another problem is the multimerization of retroviral integrases into a functional integration complex. For example, HIV-1 requires a dodecamer of interlocked integrase proteins. Integration complexes of various retroviruses have been shown to be octamers, dodecamers, and hexadecamers. This complex structure prevents the fusion of targeting protein domains to either the amino or carboxyl terminus of integrase. Thus, previous attempts to target integration by fusing a DNA binding protein to integrase have failed because of integrase multimerization or host targeting proteins.

What are needed are engineered viral integrases that can be used to insert exogenous nucleic acids into hosts. These new compositions and methods could be used, for example, to treat genetic disorders.

### III. SUMMARY

Many genetic disorders, such as cystic fibrosis (CF) or severe combined immunodeficiency (SCID), are due to the absence of a wild type gene. Gene therapy delivery of the wild type gene can be a cure to these diseases. There are additional biotechnology applications. The methods and compositions provided herein can be used with any application that benefits from stable integration at a precise site in a host genome.

Disclosed herein is an engineered retroviral integration complex comprising an engineered prototype foamy virus (PFV) integrase (IN), two or more non-naturally occurring flanking nucleic acid sequences, and a cargo nucleic acid sequence. In some embodiments, the PFV integrase comprises at least one inner PFV IN protomer and at least one outer PFV IN protomer. In some embodiments, the PFV integrase comprises two inner PFV IN protomers and two outer PFV IN protomers.

In some embodiments, the outer PFV IN promoter is engineered to comprise a nucleic acid binding domain. In some examples, a carboxyl terminus domain (CTD) region of the outer PFV IN protomer is replaced with the nucleic acid binding domain. In some embodiments, the nucleic acid binding domain is a transcription activator-like effector (TALE), a zinc finger (ZF) domain, or a Cas9/gRNA complex. The nucleic acid binding domain can target a human gene (for example, a cystic fibrosis transmembrane conductance regulator (CFTR) gene, human Alu repeats, or a portion thereof) or a nonhuman nucleic acid sequence. In some examples, the engineered PFV integrase of any preceding aspect further comprises mutations. For example, the outer PFV IN promoter comprises a D273K amino acid substitution relative to SEQ ID NO: 6, and/or the inner PFV IN protomer comprises a K120E amino acid substitution relative to SEQ ID NO: 6.

In some embodiments, the flanking nucleic acid sequences of any preceding aspect are derived from a virus (e.g., a PFV). In some embodiments, the flanking nucleic acid sequence comprises one or more blunt ends. The cargo nucleic acid sequence is flanked by the flanking nucleic acid sequences. In some examples, the cargo nucleic acid sequence is linked to at least one flanking nucleic acid sequence through a polynucleotide linker sequence. In some examples, the polynucleotide linker sequence comprises a blunt end linked to the flanking nucleic acid sequence and a sticky end linked to the cargo nucleic acid sequence.

In some embodiments, the cargo nucleic acid sequence of any preceding aspect comprises a human gene sequence or a fragment thereof (e.g., a wildtype human sequence gene or an engineered human sequence gene) or a non-human nucleic acid sequence.

Also disclosed herein is an expression vector comprising a polynucleotide sequence encoding the engineered retroviral integration complex of any preceding aspect.

Also disclosed herein is a method of expressing an exogenous nucleic acid in a subject in need thereof, comprising administering to the subject an effective amount of the engineered retroviral integration complex of any preceding aspect. In some embodiments, the subject has a genetic disorder (e.g., cystic fibrosis). In some embodiments, the subject has cancer.

Also disclosed herein is a method of increasing an expression of a protein in a subject in need thereof, comprising administering to the subject an effective amount of the engineered retroviral integration complex of any preceding aspect.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the PFV intasome structure. Left, the crystal structure of PFV integrase with viral DNA oligomers and target DNA. PFV integrase forms a tetramer. Only the CCD of the outer PFV integrase protomers is resolved. The amino terminal and carboxyl terminal domains are not visualized. Right, cryo-EM structure of a PFV intasome bound to a nucleosome. Still, the outer protomer amino and carboxyl terminal domains are not visualized.

FIG. 2 shows retroviral life cycle. Retroviral integration is stable. Retroviruses are not well controlled by innate or adaptive immunity. Different retroviruses display varied preferences for integration to genomic regions (promoters, CpG islands, etc). Site-specific integration with retrovirus is unlikely: integration complex has limited lifetime in cell drastically limiting possible search area. Probability of site specific integration by a retroviral vector is extremely low. CRISPR/Cas9 searches of genomes are successful because there are many complexes per cell.

FIG. 3 shows the dark side of integration. MLV integration favors promoters. MLV gene therapy for SCID cured 17 children. However, 3-5 years later 6 children developed leukemia. Tumors were due to integration and dysregulation of oncogenes.

FIG. 4 shows targeted retroviral integration. Fusions of DNA binding domains to integrase have failed. Fusion of chromatin binding domains to host co-factors are only successful if the endogenous gene is deleted.

FIG. 5 shows retrovirus family.

FIGS. 6A and 6B show PFV intasome structure. FIG. 6A shows crystal structure of the PFV intasome shows full length inner protomers (green, purple), the CCD of outer protomers (gold), viral DNA (yellow), and target DNA (pink, black). Structures of the outer protomer NTD and CTD domains have never been resolved. FIG. 6B shows target DNA bound by the PFV intasome is severely bent.

FIGS. 7A-7B show integration targeted to DNA damage.

FIGS. 8A-8C show deletion of the CTDs of the PFV intasome outer protomers. FIG. 8A shows that domain structure of PFV IN. PFV intasomes were assembled with outer protomers lacking either the NTDs or CTDs. FIG. 8B shows that truncation intasomes had no effect on integration to nucleosomes. FIG. 8C shows that deletion of the outer protomer CTDs significantly reduced binding to nucleosomes.

FIG. 9A shows a PFV intasome with integrase fused to zinc finger DNA binding domains at the outer protomers. The viral DNA is labeled with a fluorophore. FIG. 9B shows an agarose gel of integration products generated by wild type PFV IN or PFV IN fused to a zinc finger protein. The image is an overlay of ethidium bromide staining (red) and fluorophore fluorescence (green). The integrase zinc finger fusion is slightly less active than wild type integrase, but integration products are readily visualized. Deletion of NED/NTD had no effects. Deletion of CTD: reduces nucleosome binding, total integration efficiency is equal to FL, and integration site choice differs from FL.

FIG. 10 shows structure of ZFNs binding DNA. Multiple helical turns are bound by the ZFNs [96-98]. PFV IN-ZF may require only three ZF domains (yellow) which may be exposed on a nucleosome surface.

FIGS. 11A-11B show that PFV intasome movement is stalled by a molecular “speed bump”. FIG. 11A shows that A Cy3 labeled PFV intasome can FRET with a Cy5 labeled target DNA if it stalls at the site of DNA damage. FIG. 11B shows that DNA damage types that bend DNA but have an intact backbone (a G/T mismatch or a single T insertion) do not lead to FRET. DNA damages that allow bending and disrupt the DNA backbone (a single or two base gap, or a nick) lead to FRET indicating intasomes stalled at the damage.

FIGS. 12A-12D show PFV intasome integration to a nucleosome target. Integration products were analyzed by denaturing PAGE. FIG. 12A shows integration to a natural nucleosome positioning sequence (NPS) nucleosome. Unreacted Cy5 labeled NPS DNA is 147 bp (left lane). Integration products migrate faster than NPS DNA (blue bracket). Integration is seen only at the symmetric-36 and +36 sites in the presence of high salt (300 mM). There are more integration sites at a physiological salt concentration (100 mM). FIG. 12B shows integration to synthetic 601 NPS nucleosome has 4 major integration sites. FIG. 12C shows diagram of nucleosome with the integration sites and H2A N-termini indicated. FIG. 12D shows Cryo-EM structure of PFV intasome bound to a nucleosome. Unresolved outer protomer NED, NTD, and CTD domains in gold.

FIGS. 13A-13C show intasome delivery of cargo. FIG. 13A shows that PFV intasomes are assembled with recombinant IN and vDNA oligomers. The intasome is two inner catalytically active protomers and two outer structural protomers. Only the CCD of the outer protomers contacts the inner protomers. FIG. 13B shows an intasome with a single cargo DNA flanked by vDNA sequences. FIG. 13C shows assembly with cargo DNA flanked by vDNA empirically results in two DNAs bound.

FIG. 14 shows ligation of Cy5 labelled oligomer to PFV intasomes. A titration of a phosphorylated Cy5 labeled oligomer was added to T4 ligase and PFV intasomes (numbers indicate the molar ratio to intasomes). Reactions were incubated overnight at 14° C. Supercoiled target DNA was added to the reactions to assay for integration activity. Reactions were incubated at 37° C., deproteinated, and analyzed by agarose gel. An ethidium bromide image (gray) shows that integration activity was not affected by ligase, ligation of the Cy5 oligomer, or incubation at 14° C. The Cy5 image (red/black) shows that the Cy5 oligomer was ligated to the PFV intasomes.

FIG. 15 shows that blunt end, phosphorylated linker DNA has been successfully ligated to PFV intasome vDNA. The intasomes retain full integration activity. Cargo DNA will be ligated to distal single strand overhang ends of the linker.

FIG. 16A-16B show PFV intasome analysis. FIG. 16A shows that mass photometry is able to distinguish PFV IN monomers (PFV (IN) 1) from assembled PFV intasomes (PFV (IN) 4). FIG. 16B shows that size exclusion chromatography can purify assembled PFV intasomes. Charged moieties at the vDNA distal end (End Cy5) reduce formation of tetramers, while an internal Cy5 does not.

FIGS. 17A-17B show PFV IN-ZF intasomes. FIG. 17A shows targeted PFV intasome with an IN-ZF chimera at the outer protomers fluorophore labeled vDNA. FIG. 17B shows integration assay with unassembled IN-ZFHIv or WT IN. Image is an overlay of ethidium bromide (red) and Cy5 (green) fluorescence. IN—ZFHIV is slightly less efficient than WT IN, yet integration products are clearly visible.

FIGS. 18A-18B show PFV residues that bind target DNA or nucleosomes. FIG. 18A shows that the PFV intasome structure revealed that 7 residues of the inner protomers form hydrogen bonds with target DNA. PFV IN (R329) intercalates at the strand transfer junctions. FIG. 18B shows that the structure of a PFV intasome bound to a nucleosome identified 3 residues of the inner protomers that appear to interact with the H2A/H2B amino terminal tails. The same three residues of the outer promoter (gold) and inner protomer (purple) appear to bind the second gyre of NPS DNA.

FIG. 19 shows PFV intasome outer protomers having a targeting ZF in place of the CTD.

FIGS. 20A-20B show that CFTR cargo DNA in PFV IN-ZFCFTR intasomes can be in a (FIG. 20A) single orientation or (FIG. 20B) inverted repeats. PFV intasomes are symmetric which require an inverted repeat strategy.

FIG. 21 shows the map of expression vector pPFVINdelCTDZFfusion. It is annotated to include the hexahistidine tag, protease site, Sso7d solubility domain, PFV IN D273K delCTD, ZF domains that target the HIV genome, and an NLS.

FIG. 22 shows the map of expression vector pPFVINdelCTDTALE fusion. The plasmid comprises Sso7d solubility domain, PFV IN D273K delCTD, TALE domain that targets the CFTR gene intron 11.

FIG. 23 shows annealed viral DNA and annealed linker DNA. The sequences in FIG. 23 include SEQ ID NOs: 22-25.

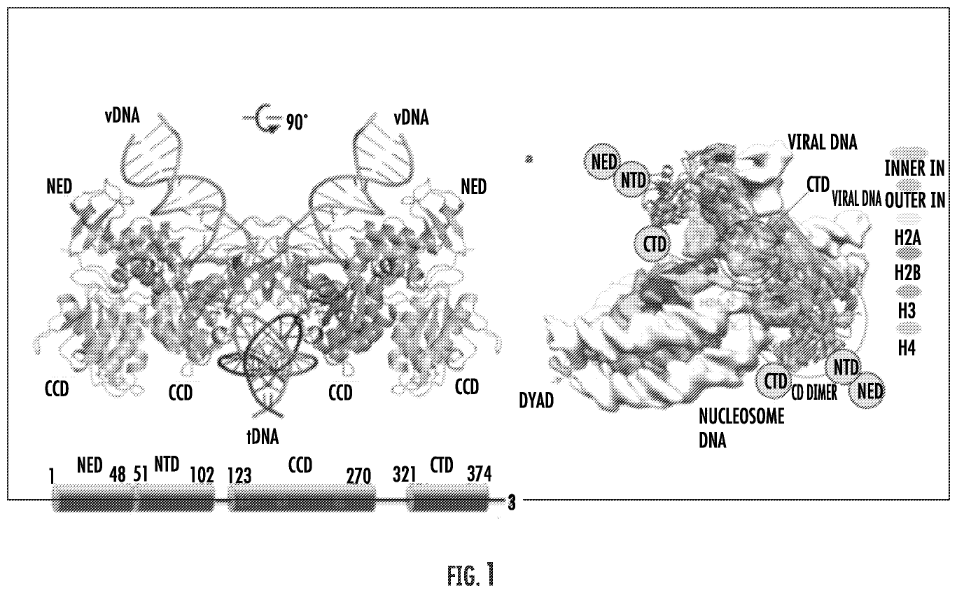

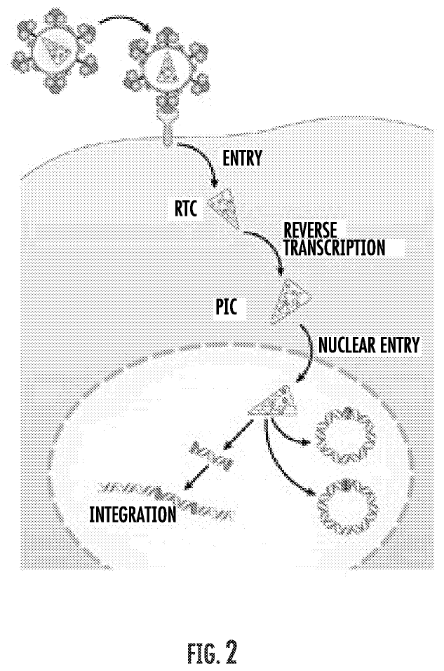

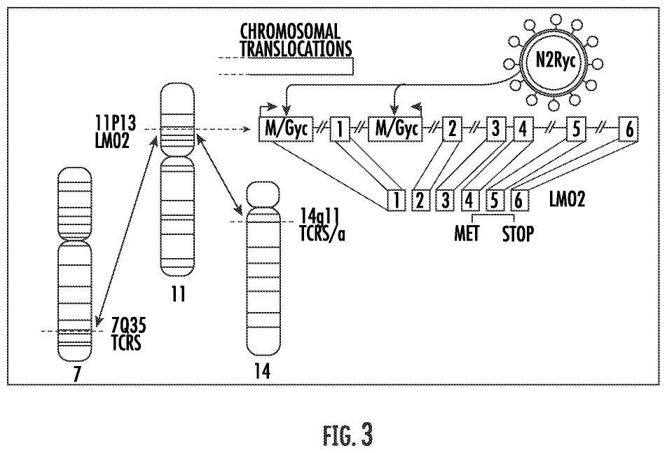

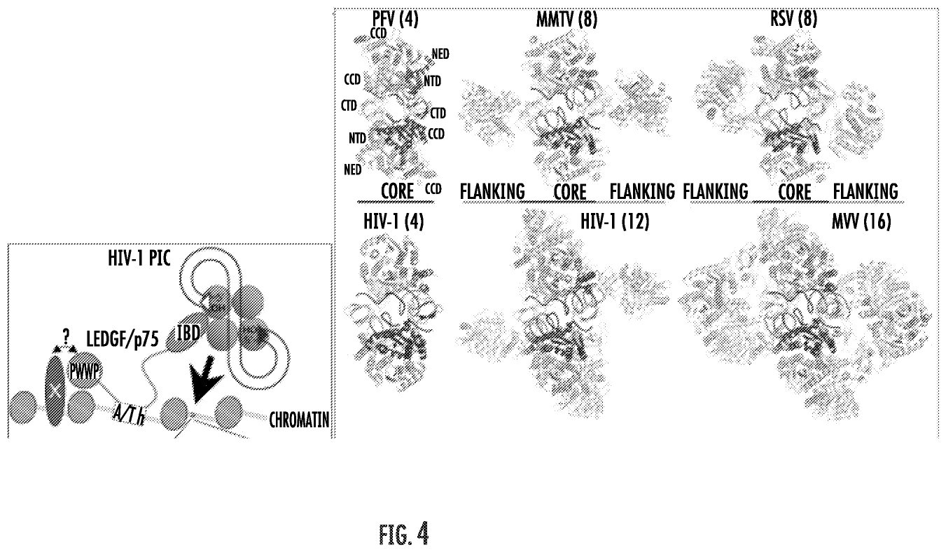

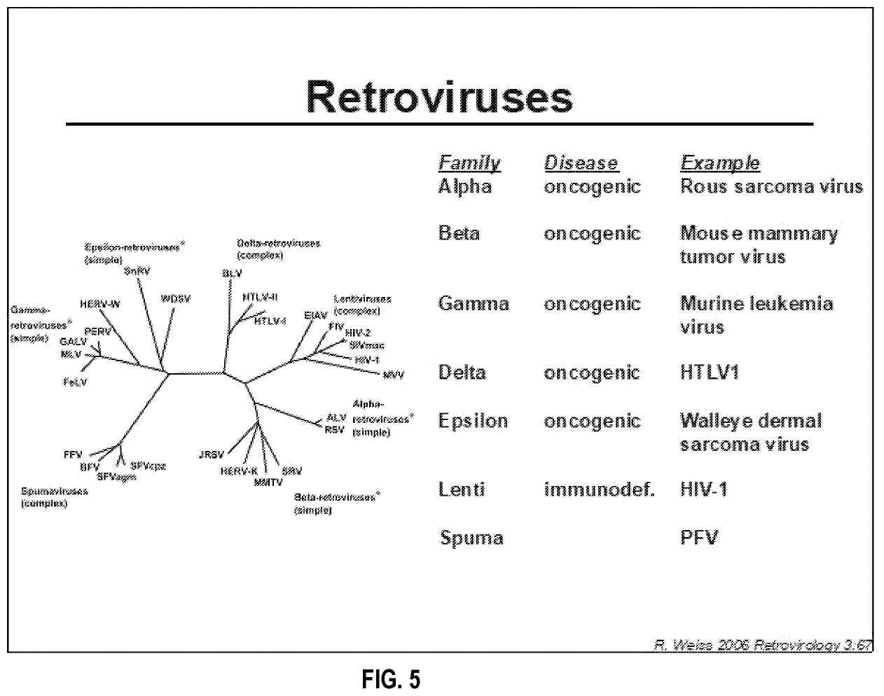

### V. DETAILED DESCRIPTION

Before the present compounds, compositions, articles, devices, and/or methods are disclosed and described, it is to be understood that they are not limited to specific methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10″as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

“Optional” or “optionally” means that the subsequently described event or circumstance may or may not occur, and that the description includes instances where said event or circumstance occurs and instances where it does not.

The term “biocompatible” generally refers to a material and any metabolites or degradation products thereof that are generally non-toxic to the recipient and do not cause significant adverse effects to the subject.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least to 25 nucleotides, at least about 75%, or at least about 90% complementary. See Kanehisa (1984) Nucl. Acids Res. 12:203.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “control” is an alternative subject or sample used in an experiment for comparison purposes.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amount.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA.

The term as used herein “engineered” and other grammatical forms thereof may refer to one or more changes of nucleic acids, such as nucleic acids within the genome of an organism. The term “engineered” may refer to a change, addition and/or deletion of a gene. Engineered cells can also refer to cells that contain added, deleted, and/or changed genes.

“Expression vector”, or “vector”, comprises a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “nucleic acid” is meant a deoxyribonucleotide or ribonucleotide polymer, which can include analogues of natural nucleotides that hybridize to nucleic acid molecules in a manner similar to naturally occurring nucleotides. In some examples, a nucleic acid molecule is a single stranded (ss) DNA or RNA molecule. In some examples, a nucleic acid molecule is a double stranded (ds) nucleic acid.

By “nucleotide” is meant the fundamental unit of nucleic acid molecules. A nucleotide includes a nitrogen-containing base attached to a pentose monosaccharide with one, two, or three phosphate groups attached by ester linkages to the saccharide moiety. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

The term “polynucleotide” refers to a single or double stranded polymer composed of nucleotide monomers.

The term “polypeptide” refers to a compound made up of a single chain of D- or L-amino acids or a mixture of D-and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/reglatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

When less than the entire sequence is being compared for sequence identity, homologs will typically possess at least 75% sequence identity over short windows of 10-20 amino acids, and can possess sequence identities of at least 85% or at least 90% or 95% depending on their similarity to the reference sequence. Methods for determining sequence identity over such short windows are described on the NCBI website.

These sequence identity ranges are provided for guidance only; it is entirely possible that strongly significant homologs could be obtained that fall outside of the ranges provided.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

67. As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “purifying” as used herein refers to purification from a biological sample, i.e., blood, plasma, tissues, exosomes, or cells. As used herein the term “purified,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

Sequence identity: The similarity between two nucleic acid sequences is expressed in terms of the similarity between the sequences, otherwise referred to as sequence identity.

Sequence identity is frequently measured in terms of percentage identity, similarity, or homology; a higher percentage identity indicates a higher degree of sequence similarity. The NCBI Basic Local Alignment Search Tool (BLAST), Altschul et al, J. Mol. Biol. 215:403-10, 1990, is available from several sources, including the National Center for Biotechnology Information (NCBI, Bethesda, MD), for use in connection with the sequence analysis programs blastp, blastn, blastx, tblastn and tblastx. It can be accessed through the NCBI website. A description of how to determine sequence identity using this program is also available on the website.

By “subject” is meant any mammal, such as humans, non-human primates, pigs, sheep, horses, dogs, cats, cows, rodents and the like. In two non-limiting examples, a subject is a human subject or a murine subject. Thus, the term “subject” includes both human and veterinary subjects.

As used herein, a “target”, “target molecule”, or “target cell” refers to a biomolecule or a cell that can be the focus of a therapeutic drug strategy, diagnostic assay, or a combination thereof, sometimes referred to as a theranostic. Therefore, a target can include, without limitation, many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof, a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset, during early onset, or after an established development of a disorder or condition. Prophylactic administration can occur for several days to years prior to the manifestation of symptoms of a disorder.

“Therapeutically effective amount” or “therapeutically effective dose” of a composition (e.g., a composition comprising an agent) refers to an amount that is effective to achieve a desired therapeutic result. In some embodiments, a desired therapeutic result is the control of a disease. In some embodiments, a desired therapeutic result is the prevention of a disease. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “variant” as used herein refers to a polypeptide or polynucleotide that differs from a reference polypeptide or polynucleotide, but retains essential properties. A typical variant of a polypeptide differs in amino acid sequence from another, reference, polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions).

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed herein are compositions, methods, systems, and kits relating to prototype foamy virus (PFV) integrase. Foamy viruses (FVs), also known as spumaviruses, belongs to the Spumaretrovirinae subfamily in the family Retroviridae. Prototype foamy virus (PFV) refers to the human virus strain isolated in the 1970s from a human nasopharyngeal carcinoma.

The integrase (IN) enzyme of prototype foamy virus (PFV) incldues four domains: amino terminal extension (NED), amino terminus (NTD), catalytic core (CCD), and carboxyl terminus domains (CTD). A PFV intasome (also herein termed as “PFV integration complex”) comprises a tetramer of INs, arranged with two inner protomers and two outer protomers. Two catalytically active inner PFV IN protomers position the vDNA ends near a target DNA, while the outer IN protomers have not been completely resolved but are commonly recognized as playing structural roles in stability of the complex.

Truncation mutants of the NTD or CTD of the outer protomers in PFV integration complexes have been characterized. The instant disclosure shows that the outer CTDs play a role in binding and targeting integration to nucleosome DNA.

In some aspects, disclosed herein is an engineered retroviral integration complex comprising a modified prototype foamy virus (PFV) integrase (IN), two or more non-naturally occurring flanking nucleic acid sequences, and a cargo nucleic acid sequence.

Disclosed herein is an engineered retroviral integration complex comprising an engineered PFV integrase which integrates an exogenous nucleic acid into a host cell genome. In some embodiments, the engineered PFV integrase comprises at least one inner PFV IN protomer and at least one outer PFV IN protomer. In some embodiments, the engineered PFV integrase comprises two inner PFV IN protomers and two outer PFV IN protomers.

The engineered retroviral integration complexes provided herein comprising at least one outer PFV IN protomer that comprises a nucleic acid binding domain are effective to target a nucleic acid sequence (e.g., a DNA sequence in host cell genome). In some embodiments, the nucleic acid sequence is a naked DNA sequence or a DNA sequence arranged in a nucleosome.

Accordingly, in some aspects, disclosed herein is an engineered retroviral integration complex comprising an engineered prototype foamy virus (PFV) integrase (IN), wherein the engineered PFV integrase comprises at least one inner PFV IN protomer and at least one outer PFV IN protomers, wherein the outer PFV IN protomer comprises a nucleic acid binding domain. In some embodiments, the engineered PFV integrase comprises two inner PFV IN protomers and two outer PFV IN protomers. In some embodiments, a carboxyl terminus domain (CTD) region of the outer PFV IN protomer is replaced with the nucleic acid binding domain.

In some embodiments, the outer PFV IN protomer comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 6 or a fragment thereof. In some embodiments, the outer PFV IN protomer comprises the sequence of SEQ ID NO: 6.

In some embodiments, the outer PFV IN protomer comprises a D273K amino acid substitution relative to SEQ ID NO: 6. In some embodiments, the outer PFV IN protomer has a deletion of CTD. In some embodiments, the outer PFV IN protomer comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 10 or 12, or a fragment thereof. In some embodiments, the outer PFV IN protomer comprises the sequence of SEQ ID NO: 10 or 12, or a fragment thereof.

In some embodiments, the outer PFV IN protomer is encoded by a polynucleotide that comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 5, 9, 11 or a fragment thereof. In some embodiments, the outer PFV IN protomer is encoded by a polynucleotide comprising the sequence of SEQ ID NO: 5, 9, 11, or a fragment thereof.

In some embodiments, the inner PFV IN protomer comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 6 or a fragment thereof. In some embodiments, the inner PFV IN protomer comprises the sequence of SEQ ID NO: 6.

In some embodiments, the inner PFV IN protomer comprises a K120E amino acid substitution relative to SEQ ID NO: 6. In some embodiments, the inner PFV IN protomer comprises a sequence at least 60% (for example, at least 60%, at least 65%, at least 70%, at least 75%, at least 80%, at least 85%, at least 90%, at least 95%, at least 96%, at least 97%, at least

98%, or at least 99%) identity to SEQ ID NO: 8 or a fragment thereof. In some embodiments, the inner PFV IN protomer comprises the sequence of SEQ ID NO: 8 or a fragment thereof.

In some embodiments, the inner PFV IN protomer is encoded by a polynucleotide that comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 5, 7 or a fragment thereof. In some embodiments, the inner PFV IN protomer is encoded by a polynucleotide comprising the sequence of SEQ ID NO: 5, 7, or a fragment thereof.

The nucleic acid binding domain provided herein can comprise any suitable nucleic acid binding domain that binds to a target site of interest. In some embodiments, the nucleic acid binding domain is a synthetically designed nucleic acid binding domain. In other embodiments, the nucleic acid binding domain is derived from a naturally occurring protein. Nucleic acid binding domain families include, for example, basic helix-loop-helix (bHLH), basic-leucine zipper, helix-turn-helix, and zinc fingers. These families exhibit a wide range of nucleic acid binding specificities and gene targets. As contemplated herein, any one of the known human transcription factor proteins can serve as a protein platform for engineering and/or reprogramming a nucleic acid binding domain to recognize a specific target site resulting in modulation of expression of an endogenous gene of interest.

The nucleic acid binding domain provided herein can be designed to recognize any target site of interest. In some embodiments, a nucleic acid binding domain is engineered to recognize a target site that is capable of modulating (e.g., upregulating or downregulating) expression from a gene of interest. In some embodiments, a nucleic acid binding domain is designed to recognize a genomic location and modulate expression of an endogenous gene. Binding sites capable of modulating expression of an endogenous gene of interest can be located anywhere in the genome that results in modulating of gene expression of the target gene. In some embodiments, the binding site is located on a different chromosome from the gene interest, on the same chromosome as the gene of interest, upstream of the transcriptional start site (TSS) of the gene of interest, downstream of the TSS of the gene of interest, proximal to the TSS of the gene of interest, distal to the gene of interest, within the coding region of the gene of interest, within an intron of the gene of interest, downstream of the poly A tail of a gene of interest, within a promoter sequence that regulates the gene of interest, within an enhancer sequence that regulates the gene of interest, or within a repressor sequence that regulates the gene of interest. In some embodiments, the nucleic acid binding domain targets a safe harbor site. The term “safe harbor site” herein refers a site in the genome able to accommodate the integration of genetic material, while not causing alteration of the host genome or promoting the expression of oncogenes or other deleterious genes. Safe harbors have been defined by 5 criteria. (i) distance of at least 50 kb from the 5′ end of any gene, (ii) distance of at least 300 kb from any cancer-related gene, (iii) distance of at least 300 kb from any microRNA (miRNA), (iv) location outside a transcription unit and (v) location outside ultraconserved regions (UCRs) of the human genome. In some embodiments, the safe harbor site is position 188,083,272 of human chromosome 1. In some embodiments, the genomic locus for integration is selected according to the method of Papapetrou and Schambach, J. Molecular Therapy, vol. 24 (4): 678-684, April 2016, which is herein incorporated by reference for the step-wise selection of a safe harbor genomic locus for gene therapy vector integration. In some embodiments, the safe harbor sites are those disclosed in U.S. Pat. No. 11,208,458, which is incorporated herein in its entirety.

In some embodiments, the nucleic acid binding domain targets a human gene. In some embodiments, the human gene is a cystic fibrosis transmembrane conductance regulator (CFTR) gene, human Alu repeats, or a portion thereof. In some embodiments, the nucleic acid binding domain targets a nonhuman nucleic acid sequence (e.g., a nucleic acid sequence of HIV).

In some embodiments, the nucleic acid binding domain targets a human CFTR gene or a portion thereof. In some embodiments, the engineered retroviral integration complex targets a promoter region, an exon, or an intron of the human CFTR gene.

In some embodiments, the human CFTR gene encodes a wildtype CFTR protein or a mutant CFTR protein. In some embodiments, the human CFTR gene encodes a mutant CFTR protein comprising a deletion of amino acid residue F508 or a mutation of amino acid residue G542 or W1282 relative to wildtype CFTR protein. In some embodiments, the human gene encoding the wildtype CFTR protein comprises the sequence of SEQ ID NO: 1. In some embodiments, the human CFTR gene comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to any of SEQ ID NOs: 1-4 or a fragment thereof. In some embodiments, the human CFTR gene comprises the sequence selected from SEQ ID NOs: 1-4 or a fragment thereof.

In some embodiments, the nucleic acid binding domain provided herein comprises a zinc finger domain, a transcription activator-like effector (TALE), or a Cas/gRNA complex.

The term “zinc finger domain” or “ZF domain” herein refers to a chimeric protein molecule comprising at least one zinc finger DNA binding domain effectively linked to at least one nuclease capable of cleaving nucleic acid sequence (e.g., DNA). Methods for generating a zinc finger are known in the art. See, e.g., U.S. Pat. Nos. 9,145,565 and 5,789,538, which is incorporated by reference herein in their entireties. In some embodiments, the ZF domain described herein does not require dimerization. In some embodiments, the ZF domain targets a nucleic acid sequence of HIV. In some embodiments, the ZF domain comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 16, 18, or a fragment thereof. In some embodiments, the ZF domain comprises the sequence of SEQ ID NO: 16 or 18 or a fragment thereof. In some embodiments, the ZF domain is encoded by a polynucleotide sequence comprising a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 15, 17, or a fragment thereof. In some embodiments, the ZF domain is encoded by a polynucleotide sequence comprising the sequence of SEQ ID NO: 15 or 17 or a fragment thereof.

In general, “Cas9/gRNA complex” herein refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence, a guide sequence, or other sequences and transcripts from a CRISPR locus. A gRNA is a component of the CRISPR/Cas system. A “gRNA” (guide ribonucleic acid) herein refers to a fusion of a CRISPR-targeting RNA (crRNA) and a trans-activation crRNA (tracrRNA), providing both targeting specificity and scaffolding/binding ability for Cas9 nuclease. A “crRNA” is a bacterial RNA that confers target specificity and requires tracrRNA to bind to Cas9. A “tracrRNA” is a bacterial RNA that links the crRNA to the Cas9 nuclease and typically can bind any crRNA. The sequence specificity of a Cas DNA-binding protein is determined by gRNAs, which have nucleotide base-pairing complementarity to target DNA sequences. Cas/gRNA complexes are known in the art. See, e.g., U.S. Pat. No. 8,697,359, which is incorporated by reference herein in its entirety.

“Transcription activator-like effector” or “TALE” herein refers to a programmable DNA-binding domain that can theoretically target any sequence. TALEs are composed of an N-terminal translocation domain, a C-terminal nuclear localization signal (NLS) with an acidic transcriptional activation domain, and a central tandem repeat nucleic acid binding domain. The TALE comprises tandem repeats of amino acid sequences (termed monomers) that are required for nucleic acid recognition and binding. TALE is known in the art. See, e.g., U.S. Pat. Nos. 10,227,581 and 11,136,566, which are incorporated by reference herein in their entireties. In some embodiments, the TALE comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 28 or a fragment thereof. In some embodiments, the TALE comprises the sequence of SEQ ID NO: 28 or a fragment thereof. In some embodiments, the TALE is encoded by a polynucleotide sequence comprising a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 29 or a fragment thereof. In some embodiments, the TALE domain is encoded by a polynucleotide sequence comprising the sequence of SEQ ID NO: 29 or a fragment thereof. In some embodiments, the TALE targets a 20 bp sequence in human CFTR gene intron 11. In some embodiments, the sequence targeted by TALE comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 32 or a fragment thereof. In some embodiments, the TALE targets the sequence of SEQ ID NO: 32.

Also, in some embodiments, the engineered retroviral integration complex disclosed herein further comprises a solubility domain to enhance the solubility of the complex. In some embodiments, the engineered retroviral integration complex further comprises a Sso7d solubility domain. In some embodiments, the solubility domain comprises Fh8, MBP, Trx, SET solubility enhancer peptide, GB1, or SUMO. In some embodiments, the solubility domain is that described in U.S. Pat. No. 10,633,662, which is incorporated herein in its entirety. In some embodiments, the Sso7d solubility domain comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 14 or a fragment thereof. In some embodiments, the Sso7d solubility domain comprises the sequence of SEQ ID NO: 14.

In some embodiments, the Sso7d solubility domain is encoded by a polynucleotide that comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 13 or a fragment thereof. In some embodiments, the Sso7d solubility domain is encoded by a polynucleotide comprising the sequence of SEQ ID NO: 13.

The engineered retroviral integration complex disclosed herein can be assembled from the modified integrases provided herein and two or more non-naturally occurring flanking nucleic acid sequences mimicking the viral nucleic acid ends. Integrase binds the viral nucleic acid ends in a sequence specific, non-covalent binding. The viral nucleic acid end is unique to each retrovirus so that the viral nucleic acid and IN must be from the same retrovirus. IN recognizes about, for example, 14 bp of the viral DNA termini. Accordingly, in some embodiments, engineered retroviral integration complex disclosed herein further comprises two or more non-naturally occurring flanking nucleic acid sequences, wherein a portion of the flanking nucleic acid sequences (which is about, for example, at least 5 bp, at least 6 bp, at least 7 bp, at least 8 bp, at least 9 bp, at least 10 bp, at least 11 bp, at least 12 bp, at least 13 bp, at least 14 bp, at least 15 bp, at least 16 bp, at least 17 bp, at least 18 bp, at least 19 bp, at least 20 bp, at least 24 bp, at least 28 bp, at least 30 bp, at least 35 bp, or at least 40 bp in length) is bound by the integrase of the engineered retroviral integration complex. In some embodiments, the flanking nucleic acid sequence is about at least 15 bp in length (including, for example, at least 16 bp, at least 17 bp, at least 18 bp, at least 19 bp, at least 20 bp, at least 24 bp, at least 28 bp, at least 30 bp, at least 35 bp, at least 40 bp, at least 45 bp, at least 50 bp, at least 55 bp, at least 60 bp, at least 70 bp, at least 80 bp, at least 90 bp, at least 100 bp, at least 120 bp, at least 150 bp, at least 180 bp, at least 200 bp, at least 250 bp, at least 300 bp, or at least 500 bp in length). In some embodiments, the flanking nucleic acid sequences are derived from a virus. In some embodiments, the flanking nucleic acid sequences are derived from PFV. In some embodiments, the flanking viral nucleic acid sequence is DNA.

In some examples, the flanking viral nucleic acid sequence is generated by a first single stranded polynucleotide annealed to a second single stranded polynucleotide. In some embodiments, the two single stranded polynucleotides are different in length. In some embodiments, the first single stranded polynucleotide comprises the sequence of SEQ ID NO: 22 or a fragment thereof. In some embodiments, the second single stranded polynucleotide comprises the sequence of SEQ ID NO: 23 or a fragment thereof. In some embodiments, the annealed polynucleotide comprises a blunt end. Accordingly, in some embodiments, the engineered retroviral integration complex disclosed herein comprises two or more flanking nucleic acid sequences, wherein the flanking nucleic acid sequences comprise at least one blunt end. In some embodiments, the flanking nucleic acid sequences comprise two blunt ends.

“Blunt end” herein refers to a nucleic acid (e.g., DNA) end where there is no unpaired bases or overhangs. “Sticky end” herein refers to a nucleic acid (e.g., DNA) end having unpaired DNA nucleotides on either 5′- or 3′-strand, which are also known as overhangs.

The assembled engineered retroviral integration complex provided herein can deliver a cargo nucleic acid sequence into a cell, wherein the cargo nucleic acid sequence comprises a nucleic acid sequence of interest the cell. The cargo nucleic acid sequence can be a DNA sequence or an RNA sequence. Accordingly, in some aspects, disclosed herein is an engineered retroviral integration complex comprising an engineered prototype foamy virus (PFV) integrase (IN), two or more non-naturally occurring flanking nucleic acid sequences, and a cargo nucleic acid sequence. In some embodiments, the cargo nucleic acid sequence comprises at least one sticky end. In some embodiments, the cargo nucleic acid sequence comprises two sticky ends.

It is herein contemplated that the cargo nucleic acid sequence is flanked by the flanking nucleic acid sequences. In some examples, the cargo nucleic acid sequence is linked to at least one flanking nucleic acid sequence through a polynucleotide linker sequence. In some embodiments, the polynucleotide linker sequence is about at least 5 bp in length (including, for example, at least 5 bp, at least 6 bp, at least 7 bp, at least 8 bp, at least 9 bp, at least 10 bp, at least 11 bp, at least 12 bp, at least 13 bp, at least 14 bp, at least 15 bp, at least 16 bp, at least 17 bp, at least 18 bp, at least 19 bp, at least 20 bp, at least 24 bp, at least 28 bp, at least 30 bp, at least 35 bp, at least 40 bp, at least 45 bp, at least 50 bp, at least 55 bp, at least 60 bp, at least 70 bp, at least 80 bp, at least 90 bp, at least 100 bp, at least 120 bp, at least 150 bp, at least 180 bp, at least 200 bp, at least 250 bp, at least 300 bp, or at least 500 bp in length). In some examples, the cargo nucleic acid sequence is linked to two flanking nucleic acid sequences each through a polynucleotide linker sequence. Accordingly, in some examples, the polynucleotide linker sequence comprises a blunt end linked to the flanking nucleic acid sequence and a sticky end linked to the cargo nucleic acid sequence. The polynucleotide linker sequence comprises a first single stranded polynucleotide annealed to a second single stranded polynucleotide. In some embodiments, the first single stranded polynucleotide comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 24 or a fragment thereof. In some embodiments, the second single stranded polynucleotide comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 25 or a fragment thereof. In some embodiments, the first single stranded polynucleotide comprises the sequence of SEQ ID NO: 24 or a fragment thereof. In some embodiments, the second single stranded polynucleotide comprises the sequence of SEQ ID NO: 25 or a fragment thereof. In some embodiments, the engineered retroviral integration complex comprises two cargo nucleic acid sequences.

In some examples, the cargo nucleic acid sequence comprises two BamHI restriction sites on both ends to be ligated to the linker sequence.

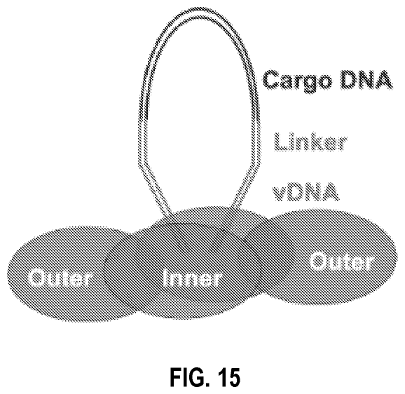

In some embodiments, the engineered retroviral integration complex provided herein comprises an engineered prototype foamy virus (PFV) integrase (IN), two non-naturally occurring flanking nucleic acid sequences, two linker sequences, and a cargo nucleic acid sequence, wherein the PFV integrase comprises two inner PFV IN protomer and two outer PFV IN protomers. In some embodiments, the engineered retroviral integration complex is as shown in FIG. 15.

The cargo nucleic acid sequence described herein can be a human gene sequence or a fragment thereof. In some embodiments, the human gene sequence is a wildtype human sequence gene or an engineered human sequence gene. In some embodiments, the cargo nucleic acid sequence comprises at least one splice signal and at least one BamHI site. In some embodiments, cargo nucleic acid sequence is from about 100 bp to about 100,000 bp in length (including, for examples, from about 150 bp to about 50,000 bp in length, from about 150 bp to about 40,000 bp in length, from about 150 bp to about 30,000 bp in length, from about 150 bp to about 20,000 bp in length, from about 150 bp to about 15,000 bp in length, from about 150 bp to about 13,000 bp in length, from about 150 bp to about 10,000 bp in length, from about 150 bp to about 8000 bp in length, from about 200 bp to about 5000 bp in length, from about 500 bp to about 4000 bp in length, from about 500 bp to around 2000 bp in length, from about 400 bp to about 2000 bp in length, from about 150 bp to around 2000 bp in length, from about 100 bp to about 2000 bp in length, from about 100 bp to about 1000 bp in length, from about 500 bp to about 5000 bp in length, from about 1000 to about 5000 bp in length, from about 1000 bp to about 10,000 bp in length, or from about 2000 bp to about 20,000 bp in length). In some embodiments, the cargo nucleic acid sequence is from about 150 bp to about 13,000 bp in length. In some embodiments, the cargo nucleic acid is about 120 bp in length, about 140 bp in length, about 160 bp in length, about 200 bp in length, about 250 bp in length, about 300 bp in length, about 400 bp in length, about 500 bp in length, about 700 bp in length, about 1000 bp in length, about 2000 bp in length, about 3000 bp in length, about 4000 bp in length, about 5000 bp in length, about 6000 bp in length, about 7000 bp in length, about 8000 bp in length, about 10,000 bp in length, about 12,000 bp in length, about 13,000 bp in length, about 15,000 bp in length, about 20,000 bp in length, about 50,000 bp in length, or about 100,000 bp in length. In some embodiments, the human gene sequence or a fragment thereof is a wildtype human CFTR sequence or a fragment thereof. In some embodiments, the wildtype human CFTR sequence is exon 12 of human CFTR gene or exon 12-27 of human CFTR gene. In some embodiments, the cargo sequence comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 19, 20, 30, 31, or a fragment thereof. In some embodiments, the cargo nucleic acid sequence comprises at least one splice signal and at least one BamHI site. In some examples, there is a about 50 bp of the intro immediately upstream of exon 12 of the CFTR nucleic acid sequence in the cargo, which includes the splice acceptor and lariat signals, an SV40 polyadenylation signal downstream of exon 27 and the CFTR stop codon. In some examples, there are two silent mutations within the sequence to remove BamHI sites present in wildtype CFTR.

112. In some embodiments, disclosed herein is an engineered retroviral integration complex comprising two outer PFV IN protomers comprising a D273K amino acid substitution relative to SEQ ID NO: 6 and two inner PFV IN protomers comprising a K120E amino acid substitution relative to SEQ ID NO: 6. In some embodiments, a carboxyl terminus domain (CTD) region of the outer PFV IN protomer is replaced with the nucleic acid binding domain. In some embodiments, the nucleic acid binding domain is a ZF binding domain targeting Alu repeats, an ING2-PHD domain targeting H3K4me3, or a ZF binding domain targeting CFTR intron 11.

113. In some embodiments, the engineered retroviral integration comprises at least one outer PFV IN protomer and at least one PFV IN inner protomer, wherein a carboxyl terminus domain (CTD) region of the outer PFV IN protomer is replaced with a TALE targeting CFTR intron 11, wherein the outer protomer comprises a D273K amino acid substitution relative to SEQ ID NO: 6, and wherein the outer protomer is linked to Sso7d solubility domain. In some embodiments, the outer PFV IN protomer comprises the sequence of SEQ ID NO: 33.

114. The engineered retroviral integration complex can be packaged into PFV retroviral vector particles and characterized for integration targeting in a host genome. Also, in some aspects, disclosed herein is a polynucleotide encoding the engineered retroviral integration complex disclosed herein. In some embodiments, the polynucleotide comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to SEQ ID NO: 21 or 34. The polynucleotide disclosed herein can be contained in a vector that can be used to deliver the polynucleotide to cells, either in vitro or in vivo. According, also disclosed herein is an expression vector comprising a polynucleotide sequence encoding the engineered retroviral integration complex disclosed herein.

115. The vectors and the delivery methods can largely be broken down into two classes: viral based delivery systems and non-viral based delivery systems. For example, the nucleic acids and the engineered retroviral integration complexes can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods are modified to specifically function with large DNA molecules. Further, these methods can be used to target certain diseases and cell populations by using the targeting characteristics of the carrier.

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed polynucleotides (e.g., a polynucleotide encoding the engineered retroviral integration complex) or the disclosed engineered retroviral integration complexes into the cell without degradation and include a promoter yielding expression of the gene in the cells into which it is delivered. Viral vectors can be, for example, Adenovirus, Adeno-associated virus, Herpes virus, Vaccinia virus, Polio virus, AIDS virus, neuronal trophic virus, Sindbis and other viruses. Also preferred are any viral families which share the properties of these viruses which make them suitable for use as vectors. A preferred embodiment is a viral vector which has been engineered so as to suppress the immune response of the host organism, elicited by the viral antigens.

In some embodiments, the viral vector is a PFV vector. In some embodiments, the vector comprises a PFV Gag (R540Q) mutation. Accordingly, in some embodiments, also disclosed herein is a PFV vector that comprises a polynucleotide encoding the engineered retroviral integration complex disclosed herein, wherein the PFV vector comprises a PFV Gag (R540Q) mutation, wherein the engineered retroviral integration complex comprises an outer PFV IN protomer comprising a D273K amino acid substitution relative to SEQ ID NO: 6 and an inner PFV IN protomer comprising a K120E amino acid substitution relative to SEQ ID NO: 6. In some embodiments, a carboxyl terminus domain (CTD) region of the outer PFV IN protomer is replaced with the nucleic acid binding domain. In some embodiments, the nucleic acid binding domain is a ZF binding domain targeting Alu repeats, an ING2-PHD domain targeting H3K4me3, or a ZF binding domain targeting CFTR intron 11. In some embodiments, also disclosed herein is a PFV vector that comprises the engineered retroviral integration complex disclosed herein, wherein the PFV vector comprises a PFV Gag (R540Q) mutation, wherein the engineered retroviral integration complex comprises an outer PFV IN protomer comprising a D273K amino acid substitution relative to SEQ ID NO: 6 and an inner PFV IN protomer comprising a K120E amino acid substitution relative to SEQ ID NO: 6. In some embodiments, a carboxyl terminus domain (CTD) region of the outer PFV IN protomer is replaced with the nucleic acid binding domain. In some embodiments, the nucleic acid binding domain is a ZF binding domain targeting Alu repeats, an ING2-PHD domain targeting H3K4me3, or a ZF binding domain targeting CFTR intron 11.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsulation, and promoters to control the transcription and replication of the viral genome. When engineered as vectors, viruses typically have one or more of the early genes removed and a gene or gene/promotor cassette is inserted into the viral genome in place of the removed viral DNA.

In some embodiments, the polynucleotide disclosed herein is contained in an adeno-associated virus (AAV) vector. This defective parvovirus is a preferred vector because it can infect many cell types and is nonpathogenic to humans. AAV type vectors can transport about to 5 kb and wild type AAV is known to stably insert into chromosome 19. Vectors which contain this site-specific integration property are preferred. The AAV vector can further comprise the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The disclosed vectors thus provide DNA molecules which are capable of integration into a mammalian chromosome without substantial toxicity. The inserted genes in viral and retroviral can contain promoters, and/or enhancers to help control the expression of the desired gene product.

The disclosed engineered retroviral integration complexes or the polynucleotides encoding the integration complex can be delivered to the target cells in a variety of ways. For example, the engineered retroviral integration complexes or the polynucleotides can be delivered through electroporation, or through lipofection, or through calcium phosphate precipitation. The delivery mechanism chosen will depend in part on the type of cell targeted and whether the delivery is occurring for example in vivo or in vitro.

124. Thus, disclosed herein are compositions comprising, in addition to the disclosed the engineered retroviral integration complexes, polynucleotides or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

Liposomes can be made using any method, e.g., as described in Park, et al., U.S Publication No. 20070042031, incorporated herein by reference in its entirety, including method of producing a liposome by encapsulating a composition disclosed herein, the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In the methods described above which include the administration and uptake of the engineered retroviral integration complex or exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed engineered retroviral integration complex, nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

In some embodiments, method of delivery agents (e.g., proteins or nucleic acids) is that described in U.S. Pat. No. 10,829,787, which is incorporated herein in its entirety.

The nanoparticle used herein can be any nanoparticle useful for the delivery of nucleic acids or the engineered retroviral integration complex. The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. In some embodiments, the nanoparticle comprises a lipid-like nanoparticle. See, for example, WO2016187531A1, WO2017176974, WO2019027999, or Li, B et al. An Orthogonal array optimization of lipid-like nanoparticles for mRNA delivery in vivo. Nano Lett. 2015, 15, 8099-8107; which are incorporated herein by reference in their entirety.

Nanoparticles disclosed herein include one, two, three or more biocompatible and/or biodegradable polymers. For example, a contemplated nanoparticle may include about 10 to about 99 weight percent of a one or more block co-polymers that include a biodegradable polymer and polyethylene glycol, and about 0 to about 50 weight percent of a biodegradable homopolymer. Polymers can include, for example, both biostable and biodegradable polymers, such as microcrystalline cellulose, hydroxypropyl cellulose, hydroxypropyl methylcellulose, polyalkylene oxides such as polyethylene oxide (PEG), polyanhydrides, poly(ester anhydrides), polyhydroxy acids such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

130. Also disclosed herein is an engineered cell comprising the engineered retroviral integration complex disclosed herein.

### C. Methods of Treatment

131. Disclosed herein is a method of expressing an exogenous nucleic acid in a subject in need thereof, comprising administering to the subject an effective amount of the engineered retroviral integration complex disclosed herein.

132. In some aspects, disclosed herein is a method of expressing an exogenous nucleic acid in a subject in need thereof, comprising administering to the subject an effective amount of the engineered retroviral integration complex comprising an engineered prototype foamy virus (PFV) integrase (IN), two or more non-naturally occurring flanking nucleic acid sequences, and a cargo nucleic acid sequence, wherein the cargo nucleic acid is expressed in the subject.

133. Also disclosed herein is a method of increasing an expression of a protein in a subject in need thereof, comprising administering to the subject an effective amount of the engineered retroviral integration complex disclosed herein.

In some aspects, disclosed herein is a method of increasing an expression of a protein in a subject in need thereof, comprising administering to the subject an effective amount of an engineered retroviral integration complex comprising an engineered prototype foamy virus (PFV) integrase (IN), two or more non-naturally occurring flanking nucleic acid sequences, and a cargo nucleic acid sequence, wherein the cargo nucleic acid comprises a nucleic acid sequence encoding the protein. The extent of effect of increasing or enhancing an expression of the protein in the subject is relative to a control (e.g., a subject not being administered with the composition).

In some embodiments, the subject has a genetic disorder. Accordingly, also disclosed herein is a method of treating, preventing, and/or mitigating a genetic disorder, comprising administering to the subject a therapeutically effective amount of the engineered retroviral integration complex disclosed herein that targets a human gene. Genetic disorder involves a gene having a mutation that leads to improper translation thereof. The improper translation produces a dysfunctional protein, causes a reduction or abolishment of synthesis of a protein, or causes over-production of a protein. In some embodiments, the genetic disorder is associated with single nucleotide polymorphisms. In some embodiments, the genetic disorder is associated with deletion of a gene.

In some embodiments, the genetic disorder is cystic fibrosis. Accordingly, in some aspects, disclosed herein is a method of treating cystic fibrosis in a subject in need thereof, comprising administering to the subject a therapeutically effective amount of the engineered retroviral integration complex disclosed herein that targets a human CFTR gene or a portion thereof. In some embodiments, the engineered retroviral integration complex targets the promoter region, the intron, or the exon of the human CFTR gene. In some embodiments, the ZF binding domain of the engineered retroviral integration complex targets intron 10 of the human CFTR gene. In some embodiments, the subject has a mutated CFTR gene (e.g., the mutated human CFTR gene encodes a mutant CFTR protein comprising a deletion of amino acid residue F508 or a mutation of amino acid residue G542 or W1282 relative to wildtype human CFTR protein). In some embodiments, the human CFTR gene comprises a sequence at least 60% (for example, at least 60%, at least 65%, at least 70%, at least 75%, at least 80%, at least 85%, at least 90%, at least 95%, at least 96%, at least 97%, at least 98%, or at least 99%) identity to any of SEQ ID NOs: 1-4 or a fragment thereof. In some embodiments, the human CFTR gene comprises the sequence selected from SEQ ID NOs: 1-4.

137. In some embodiments, the genetic disorder is associated with deletion of a gene. In some embodiments, the cargo nucleic acid sequence of the engineered retroviral integration complex comprises the gene sequence or a fragment thereof. In some embodiments, the engineered retroviral integration complex targets a safe harbor site.

138. Accordingly, the method disclosed herein can treat, decrease, mitigate, and/or prevent cystic fibrosis and/or a symptom thereof (e.g., lung infections or pneumonia, coughing with thick mucus, wheezing, bulky, greasy bowel movements, and/or trouble gaining weight or poor height growth). It should be understood and herein contemplated that the extent of effect of treating, decreasing, mitigating, and/or preventing cystic fibrosis and/or a symptom thereof is relative to a control (e.g., a subject not being administered with the composition).

139. It is understood and herein contemplated that the timing of a genetic disorder onset can often not be predicted. The disclosed methods of treating, preventing, reducing, and/or inhibiting a genetic disorder can be used prior to or following the onset of a genetic disorder. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2, or 1 hour prior to onset of a genetic disorder; or 1, 2, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 days, 3, 4, 5, 6, 7, 8, 9, 10, 11, 12 months, 2, 3, 4, 5, 6, 7, 8, 9, 10, 11, 12, 15, 18, 24, 30, 36, 48, 60 or more years after onset of a genetic disorder.

140. The compositions described herein may be in any appropriate dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intravenous, intrathecal, intravitreal, intracerebral, gingival, subgingival, intracerebroventricular, and intradermal. Such formulations may be prepared by any method known in the art.

### D. Methods of Generating the Complex

141. Also disclosed herein is a method of making an engineered prototype foamy virus (PFV) integration complex, comprising
a) expressing an engineered PFV integrase,
b) purifying the engineered PFV integrase,
c) contacting the purified engineered PFV integrase with a non-naturally occurring flanking nucleic acid sequence, thereby creating a complex comprising the engineered PFV integrase and the flanking nucleic acid sequence;
d) ligating a linker sequence to the flanking nucleic acid sequence of the complex of step c); and
e) ligating a cargo nucleic acid sequence to the linker sequence of step d);

142. In some embodiments, the flanking nucleic acid sequences are derived from a virus (e.g., PFV). In some embodiments, the flanking nucleic acid sequences comprise at least one a blunt end. In some embodiments, the flanking nucleic acid sequences comprise two blunt ends. In some embodiments, the cargo nucleic acid sequence comprises at least one sticky end. In some embodiments, the cargo nucleic acid sequence comprises two sticky ends. In some embodiments, the cargo nucleic acid sequence is flanked by the flanking nucleic acid sequences. In some embodiments, the cargo nucleic acid sequence is linked to at least one flanking nucleic acid sequence through a polynucleotide linker sequence. In some embodiments, the cargo nucleic acid sequence is linked to two flanking nucleic acid sequences each through a polynucleotide linker sequence. In some embodiments, the polynucleotide linker sequence comprises a blunt end linked to the flanking nucleic acid sequence and a sticky end linked to the cargo nucleic acid sequence. In some embodiments, the polynucleotide linker sequence comprises a restriction digestion side.

### VI. EXAMPLE: SEQUENCE-TARGETED RETROVIRAL INTEGRATION FOR THE DELIVERY OF CORRECTIVE TRANSGENES

143. Engineered purified retroviral integration complexes can be targeted to specific genetic regions allowing complementation of disease-associated mutations at endogenous loci.

144. Disclosed herein are engineer retroviral integration complexes, termed intasomes, that can be delivered to cells. The complexes are targeted to integrate at the CFTR locus by zinc finger domains. In a single step, the wild type CFTR sequence is integrated and under control of endogenous elements. Importantly, the integration complexes have no viral sequences or exogenous promoters that can be oncogenic. Develop sequence-specific targeted recombinant intasomes.

145. All previous attempts to target retroviral integration have failed for a variety of reasons including disruption of the integrase multimer, innate targeting by host integration co-factors, and limited search capacity of viral integration complexes. Inspired by the successful site-specific targeting of CRISPR/Cas9 ribonucleic particles (RNPs), provided herein are engineer retroviral integration complexes for similar targeting and delivery to cells. This approach allows the molar excess of complexes required for a successful search of genomic DNA, as observed with Cas9 RNPs.

146. A. Ligation of a cargo DNA into purified prototype foamy virus (PFV) intasomes. Intasomes are assembled with short oligomer DNAs mimicking the viral DNA ends. Intasomes delivering corrective transgenes must deliver a single cargo DNA encoding the relevant wild type sequence. Intasome assembly with a single long DNA is empirically not possible. Provided herein are the first to demonstrate ligation of additional DNAs to the vDNA ends following intasome assembly. The resulting intasomes retain activity following ligation. The cargo DNA encoding CFTR exons is subsequently ligated to the linker oligomers.

147. B. Sequence specific integration of ZF targeted PFV intasomes in vitro. PFV intasomes perform site-specific integration when a molecular “speed bump” in the form of DNA damage is added to the target. This observation is adapted to a molecular “anchor” on the PFV intasomes. This is a site-specific zinc finger protein added to the outer intasome protomers in place of the integrase C-terminal domain. The targeted intasomes are assayed by fluorescence resonance energy transfer (FRET) with a single molecule total internal reflection fluorescence (TIRF) microscope to demonstrate stalling at the ZF binding site. Targeting is also assayed by integration to targets with and without the ZF binding site. Mutations of PFV integrase are analyzed for reduced off-target binding and integration. Evaluate the integration profiles of PFV IN chimeras in cells.

148. Purified intasomes are delivered to cells with techniques adapted from CRISPR RNP delivery. The ability to target PFV integration and reduce off-target insertion is assayed in cells with retroviral vectors and intasomes.

149. A. Profile the integration sites of IN mutants delivered by viral vector particles. Targeting a unique genomic site with retroviral vector particles remains a challenge because integration complexes have a limited search area. Targeted integration during retroviral transduction is assessed by increasing the number of target sites per genome. PFV integrase is fused to a ZF targeting Alu repeats (>150,000/cell), ING-PHD domain (>27,000/cell), and a ZF targeting the CFTR locus (2/cell). PFV vectors is assayed for integration efficiency and integration sites are mapped by Next Generation Sequencing (NGS).

150. B. Deliver PFV intasomes to cells by lipofection and electroporation and profile integration sites. Similar to techniques used with Cas9 RNPs, engineered PFV intasomes with a GFP cargo are added to cells by lipofection and electroporation. Integration efficiency is quantified by GFP fluorescence and integration sites are mapped by NGS.

151. C. Determine expression of exogenous CFTR exons in cells of interest. PFV intasomes targeted to the CFTR locus by ZF domains are assembled with cargo DNA encoding CFTR exons. The cargo is a single exon 12 that rescues the G542X mutation or a “superexon” encoding exons 12-27. These PFV intasomes are transfected to isogenic cells that are homozygous for CFTR mutations F508del, G542X, or W1282X. The rescue of full length CFTR expression in G542X cells is monitored by Western analysis.

### Background and Science.

152. Cystic fibrosis (CF) is an autosomal disorder affecting 1 in 2000-3000 births among Caucasians. The disease is caused by over 2000 different mutations of the cystic fibrosis trans-membrane conductance regulator (CFTR) gene expressed in epithelial cells of multiple organs. The CFTR mutations can be grouped as 6 different classes based on the type of defect. The most common mutation CFTR (F508del) affects ˜70% of patients. Three oral drugs-ivacaftor, lumacaftor, and tezacaftor-can be used to treat patients who express a defective CFTR protein. These drug therapies cannot rescue Class I mutations that abolish CFTR protein expression, such as G542X. Small molecules that induce translational read-through can suppress the CFTR early termination mutants. However, it is difficult to predict the read-through sequence outcomes which may display reduced activity. Many CF patients, including those who cannot tolerate the drugs, require a cure with a gene therapy approach that delivers the wild type CFTR sequence.

153. While some genetic diseases can be successfully treated by the introduction of a constitutive promoter and transgene at any site in the patient genome, diseases such as CF require more sophisticated control of complementing transgene expression. Ideally, rescue with a wild type CFTR gene in CF patients would include control by the native promoter. Indeed, regulation of CFTR transcription remains poorly understood. Targeted retroviral integration of wild type CFTR sequences to the endogenous locus is one possible approach to curing CF patients.

154. Retroviruses are attractive gene therapy vectors due to their relatively large coding capacity for transgenes, low immunogenicity, and stable integration. These viruses are defined by two activities: reverse transcription and integration. Reverse transcriptase copies the viral genomic RNA into a linear double stranded cDNA. Following reverse transcription, the pre-integration complex (PIC) consists of integrase (IN) protein bound to the ends of the viral cDNA and a poorly characterized set of viral and cellular proteins. IN cleaves 2 nucleotides from the 3′ ends of the cDNA and mediates covalent joining of the recessed 3′ hydroxyls to the host genomic DNA. The product of integration is the viral genome flanked by 4-6 base gaps of host DNA and 5′ dinucleotide flaps of viral cDNA. Host DNA repair completes the integration reaction resulting in the integrated provirus. Retrovirus proviral genomes vary in size; the human immunodeficiency virus (HIV-1) genome is ˜10 kb and the prototype foamy virus (PFV) genome is ˜13 kb.

155. There are seven genera of retroviruses including the oncogenic retroviruses alpha through epsilon, the lentiviruses which cause immunodeficiency, and the spumaviruses (also called foamy viruses, FV) which do not lead to any disease in their host. Gene therapy with oncogenic gamma retrovirus murine leukemia virus (MLV) based vectors led to leukemia in several patients. The tumors were shown to be a direct result of integration at the promoters of known oncogenes and their dysregulation. MLV is known to have a strong preference for integration near transcription start sites (Table 1). The propensity for integration associated pathogenesis makes any oncogenic retrovirus a poor choice for gene therapy applications.

Table 1. Retroviral integration site preferences for genomic elements. HIV, MLV, and PFV integration site preferences were compared to matched random controls and are shown as a likelihood percentage. The random likelihood of integration to transcription units (TU) is 45.2%, but 82.3% of HIV-1 integration sites are seen in TUs. PFV integration appears to favor transcription start sites (TSS, +2 kb) and CpG islands. However, these preferences are due to PFV Gag tethering of IN to chromatin. This preference is abolished by Gag (R540Q) revealing that PFV IN has no intrinsic preference for any genomic features.

| | TU | TSS | CpG |
|---|---|---|---|
| Random Control | 45.2 | 4.2 | 4.2 |
| HIV | 82.3 | 4.5 | 5.8 |
| MLV | 57.6 | 47.0 | 50.5 |
| PFV | 35.8 | 9.0 | 8.9 |
| PFV Gag(R540Q) | 35.6 | 3.3 | 3.7 |

156. Lentiviral vectors (LVV) have been used successfully in gene therapy trials for several diseases including Wiskott-Aldrich syndrome, metachromatic leukodystrophy, beta-thalassemia, and X-linked adrenoleukodystrophy. Lentiviruses are unique among retroviruses in their ability to transduce non-dividing cells. Typical LVV gene therapy involves transduction of patient cells ex vivo and subsequent re-infusion to the patient. LVV have been used to deliver a CFTR transgene driven by the adenovirus Ela promoter to lungs of mice, rats, and marmosets. While there are questions regarding the relevance of some of these animal models to human CF disease, greater questions surround the use of LVV in patients. In 2021 two LVV gene therapy trials conducted by Bluebird Bio were halted due to observed adverse events in patients. In one case, it was determined that LVV integration did not lead to the adverse effects observed in two patients treated for sickle cell anemia and this trial resumed. The second LVV trial halted in August 2021, was a treatment for the rare neurological disease cerebral adrenoleukodystrophy. In this case, a pre-leukemic disorder, myelodysplastic syndrome (MDS), was diagnosed in one patient. Further examination revealed LVV integration at a site associated with MDS. Two additional patients appear to have a similar pathology. The transcription dysregulation of the MDS associated gene may be partially due to the promoter used in this LVV. While LVV have been successfully used in gene therapy for hundreds of patients with no adverse effects, these new results underscore that integration at transcriptionally active regions by LVV remains a significant concern for patient safety.

157. FVs are less studied than other retroviral genera, possibly due to the lack of any pathology associated with infection. Simian FV are highly prevalent in their natural hosts leading to significant zoonotic infection of hunters and zookeepers; remarkably, none of the FV infected humans have become ill, which is atypical for xenotropic viral infections. The FV life cycle differs from other retroviruses by budding new virus particles into the endoplasmic reticulum, as opposed to budding from the plasma membrane. This unusual aspect of the FV life cycle precludes pseudotyping FV vector (FVV) particles with alternative envelope proteins, such as vesicular stomatitis virus G protein (VSV-G) which is commonly used to pseudotype LVV. The cellular receptor bound by the FV envelope protein has not been identified making the tropism of these viruses somewhat mysterious, yet it does appear to be promiscuous for target cells. Studies of cultured cells have shown fibroblasts, epithelial cells, and lymphocytes can be infected by FV. FV can be cytopathic in cultured cells, but this has not been observed during infection of animals or humans. Importantly, gene therapy FVV have been successfully used in dogs and mice with no signs of oncogenic transformation in any of the animals. Unique among retroviral gene therapy vectors, FVV have been administered to dogs intravenously instead of ex vivo. These FVV were not pseudotyped to target a particular cell population, but successfully transduced hematopoetic stem/progenitor cells for the treatment of canine X-SCID.

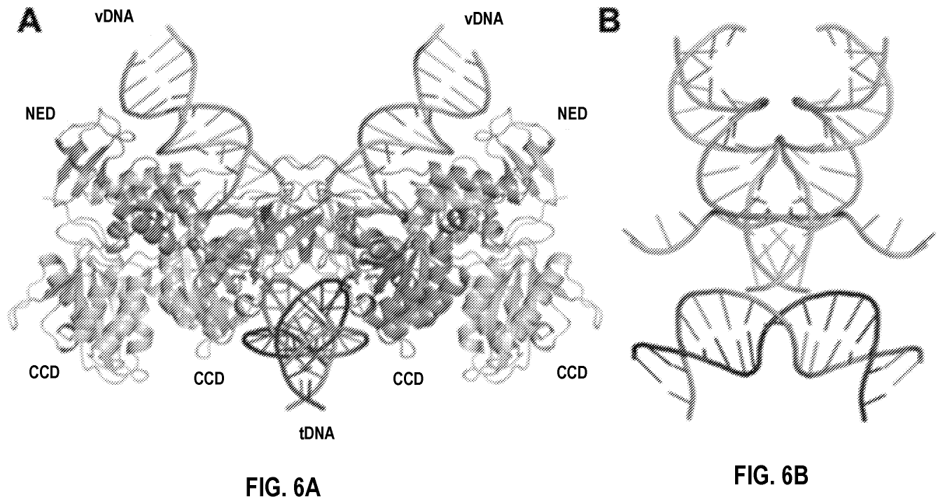

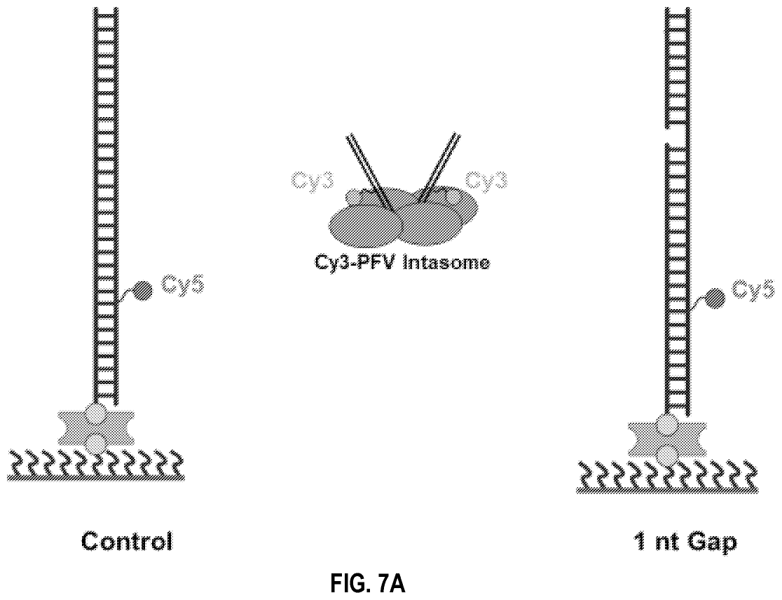

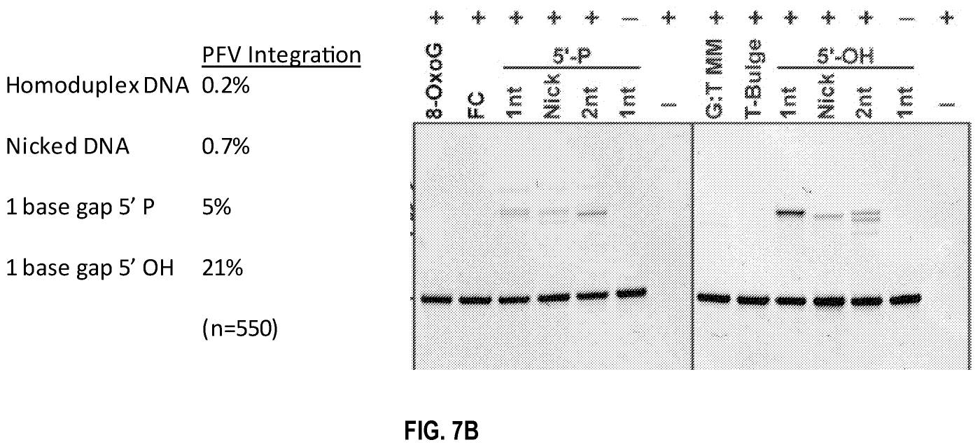

158. Since the 1990s several attempts have been made to target retroviral integration by adding DNA binding domains to IN. Most of these studies have employed HIV-1 IN with a minority attempting to target avian sarcoma leukosis virus (ASLV) IN. All of these attempts failed both in vitro and in vivo due to an incomplete understanding of retroviral integration dynamics and PIC structure. Since 2010 it has been shown that retroviral INs assemble as multimers. For example, the alpharetrovirus Rous sarcoma virus IN forms an octamer, the lentivirus Maedi visna virus IN forms a hexadecamer, the lentivirus HIV-1 may form a dodecamer, and the spumavirus PFV IN forms a tetramer. The first integration complex, termed an intasome, to be visualized was a tetramer of PFV IN assembled with short DNA oligomers mimicking the viral cDNA ends (vDNA) (FIG. 6A). The intasome consists of recombinant IN assembled with vDNA and differs from the viral PIC by the absence of full-length cDNA and any other proteins. The outer protomer amino (NTD) and carboxyl (CTD) terminal domains have never been structurally resolved; only the catalytic core domain (CCD) has been observed. The NTD and CTD of the outer protomers can be deleted from intasomes without affecting integration activity. The revelation that many retroviral IN complexes are multimers greater than a tetramer offers a partial explanation for previous failures to target retroviral integration. For example, adding DNA binding domains to HIV-1 IN disrupted the formation of the putative dodecamer complex. The higher order multimers of lentiviral INs indicates that targeting LVV integration is be feasible. However, the tetrameric PFV intasomes is highly amenable to fusions of DNA binding domains to the outer protomers and targeting of integration site choice in the host genome.

159. In addition to the complexity of multimeric IN complexes, many retroviruses also employ a host protein as an integration co-factor. HIV-1 PICs bind to the host transcription activator lens epithelium derived growth factor (LEDGF/p75) which directs integration to actively transcribed genes and stabilizes the integration complex. It has been possible to re-direct HIV-1 integration to alternative sites with chimeric LEDGF/p75 proteins. However, this strategy requires the absence of endogenous LEDGF/p75, a ubiquitously expressed protein. Deletion of endogenous LEDGF/p75 is not feasible in patients. PFV does not require a host co-factor for integration, adding to the appeal of targeting PFV IN.

160. PFV IN is attractive for targeted integration for several reasons:
PFV is not associated with any known disease in the natural hosts nor xenotropic infections, indicating that the use of PFV IN is safe. This is not true for any other retrovirus genus.
The tetrameric PFV intasome configuration readily permits fusions to the outer protomers without disruption of the multimeric organization.
There are no host co-factors that naturally direct PFV integration that can confound targeting. Human T cell leukemia virus (HTLV-1) retroviral intasomes are tetrameric, but this retrovirus requires a host co-factor for integration.
PFV PICs remain stable in the cytoplasm for several weeks allowing successful integration in slowly dividing cells.
Purified PFV intasomes require only 14 bp of viral sequence and do not encode promoters or enhancers that can dysregulate host oncogenes near the integration site.
Delivery of purified intasomes precludes the possibility of reverse transcriptase induced mutations.

Targeting unique sites in the human genome

161. Targeted retroviral integration with retroviral or lentiviral vector particles that recapitulate the viral life cycle from entering the cell through integration is unlikely to be efficient or clinically relevant. The retroviral life cycle is inefficient at every step. Only ˜10% of complete HIV-1 or MLV cDNA becomes integrated proviruses. This indicates that an excess of vector particles is required to achieve a single PIC per nucleus. Tracking of HIV-1 PICs during infection shows a limited search area in the nucleus. The probability is vanishingly low that any single PIC finds a unique targeted site during that limited search. Vector particle-based delivery of any novel sequence is extremely unlikely to generate targeted integration at a clinically relevant frequency.

162. Genome editing technologies, such as zinc finger nucleases (ZFNs) and clustered regularly interspaced short palindromic repeats (CRISPR), have demonstrated that sequence-based targeting in a genome is possible. These editors are often delivered by plasmid transfection or lentiviral vector transduction, both of which generate exceptionally large numbers of the proteins or complexes in a cell. Alternatively, purified recombinant CRISPR ribonucleoprotein complexes (RNPs) comprised of recombinant Cas9 and a synthetic guide RNA can be delivered to cells by lipofection or electroporation, which can also deliver a large number of complexes. For example, the protocol for the Alt-R CRISPR-Cas9 system sold by Integrated DNA Technologies calls for more than 107 Cas9 RNPs/cell in a lipofectamine based transfection and more than 108 RNPs/cell during electroporation. The actual number of RNPs searching each genome is affected by the transfection efficiency, yet this demonstrates the molar excess of RNPs per cellular genome. These genome editors are able to find a single genomic site due to the great excess of proteins or complexes per nucleus. Yet only a single ZFN pair or CRISPR RNP is required to generate a double strand break, while the vast majority of the complexes present in a cell will not find their target sequence and will be degraded. Transduction with a retroviral vector is incapable of delivering a similar magnitude of PICs to search a genome.

163. While ZFNs and CRISPR can locate a sequence specific site in the context of a human genome, their functionality is limited. These genome editors can efficiently generate a double strand break, but they rely on host DNA repair pathways for actual editing. Genome editing is often used to abolish expression of a gene. In this case, the error-prone non-homologous end joining (NHEJ) pathway inserts or deletes base pairs at the repair junction. Genome editing is far less efficient at introducing new desired sequences to the host genome. These strategies require the delivery of a DNA template encoding the novel sequence and the activity of homologous recombination (HR) DNA repair. The HR pathway is only active during S phase of the cell cycle, thus requiring that the cells are actively replicating. The efficiency of introducing novel sequences into the host genome by homology dependent repair (HDR) is low.

HDR approaches with ZFNs and CRISPR have been applied to the CFTR gene with limited success. For example, a ZFN pair targeting intron 10 and an HDR donor DNA encoding wild type exon 11 were added to cells with the F508del mutation in exon 11. The sequence was repaired to wild type, but with 1% efficiency.

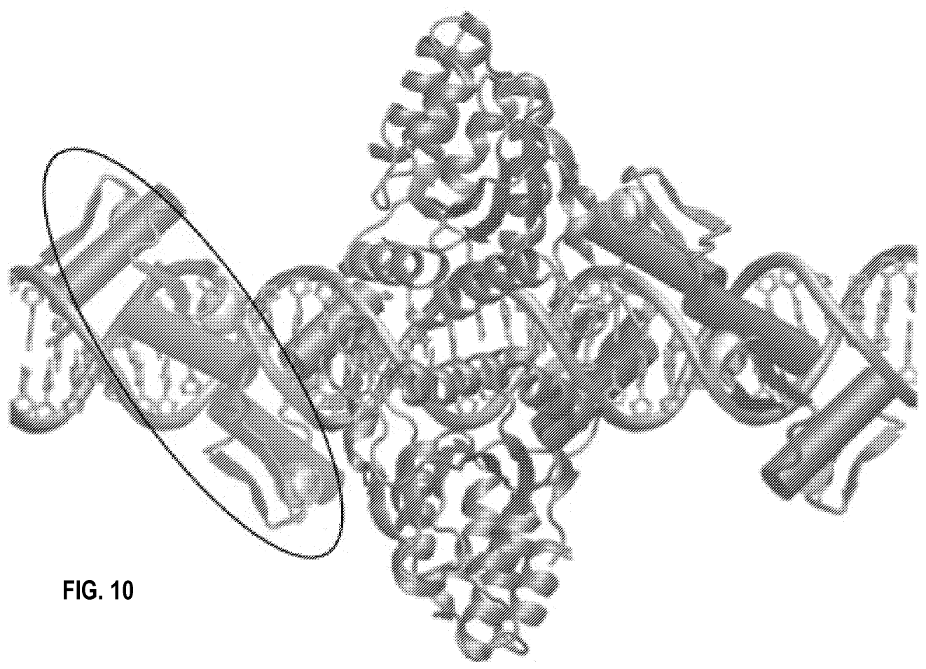

In some cases, it was proposed that nucleosome occupancy at the CFTR gene prevented efficient binding of the two ZFNs. ZFNs must bind several turns of the DNA helix without being blocked by the histone octamer to achieve efficient binding and endonucleolytic cleavage (FIG. 10). ZFNs typically require at least 6 unique ZF domains, 3 in each ZFN. In contrast, only 3 ZF domains are required for intasome targeting to a unique genomic sequence. Since intasomes are structurally symmetric, the same ZF protein are fused to both PFV IN outer protomers. Based on structural studies of a PFV intasome bound to a nucleosome, only one of the ZF proteins can direct integration. Unlike the obstruction of ZFNs by nucleosomes, retroviral INs preferentially integrate to nucleosome bound DNA. Previous HDR attempts to rescue wild type CFTR expression provide important insights and valuable reagents, including ZF proteins that recognize the CFTR locus. In comparison to genome editing methods that require the nuclease, donor DNA, and host DNA repair, retroviral integration is a single step reaction resulting in a stably integrated exogenous sequence

Sequence targeted retroviral integration requires large numbers of integration complexes being delivered to cells. This can be achieved with purified PFV intasomes and delivery mechanisms similar to CRISPR RNPs. Using lipofection and electroporation demonstrates delivery of integration complexes to cells. Recombinant PFV intasomes can be purified in sufficient quantities necessary to search cellular genomes and delivered to cultured cells by lipofection or electroporation. However, several novel alterations to the PFV intasomes are necessary. The data herein show sequence-targeted PFV intasomes encoding a relevant transgene.

### Results.

### Mechanisms of PFV Intasome Searches of Chromatin

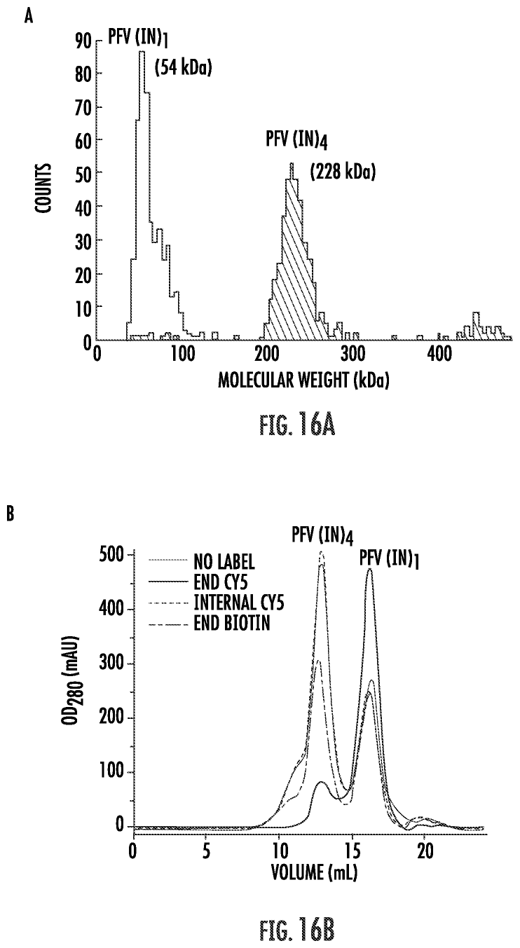

167. In order to assess targeting retroviral integration, the search dynamics of PFV intasomes was characterized by several approaches. PFV intasomes were assembled with recombinant IN and vDNA with an internal Cy3 or Cy5 fluorescent moiety. Covalently adding the fluorophore to an internal site of the vDNA does not inhibit PFV intasome assembly, unlike at the vDNA exposed end (FIG. 16B). Single molecule total internal reflection fluorescence (smTIRF) microscopy was used to visualize the interaction of PFV intasomes with 23 kb naked DNA in real time. At physiologically relevant buffer conditions, PFV intasomes have a lifetime of 2.1 seconds and search 1.6 kb of target DNA. A constant diffusion coefficient at multiple salt concentrations indicates that PFV intasomes are in continuous contact with the DNA backbone throughout the search, akin to a nut moving on a screw as opposed to a washer. These experiments revealed that PFV intasomes only rarely perform integration on linear naked DNA.

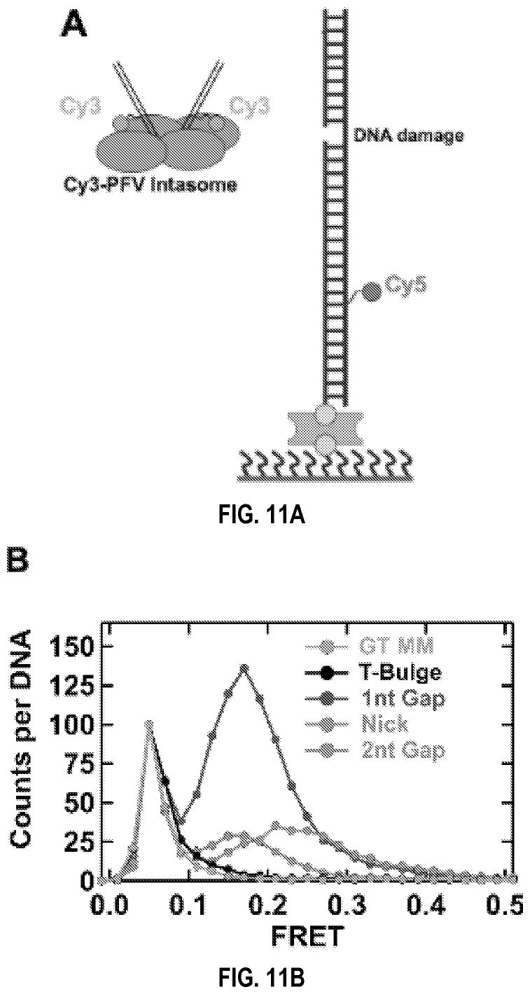

168. One reason for the rarity of PFV integration was the speed of the complexes during a search. The static PFV intasome structure revealed that the target DNA is sharply bent into the enzyme active sites (FIG. 6B). Whether a bent DNA or discontinuous DNA backbone can pause the intasome and elicit integration was tested. A 60 bp DNA substrate was engineered with an internal Cy5 fluorophore. When Cy3 labelled PFV intasomes search the 60 bp target DNA, a fluorescence resonance energy transfer (FRET) signal can be visualized by smTIRF (FIG. 11A). When a homoduplex DNA was analyzed by this method, short FRET signals were seen as the Cy3 PFV intasomes traversed the Cy5 site. The 60 bp target DNA was also engineered to have a variety of DNA damages 15 bp from the Cy5 label (FIG. 11A). Bent substrates, such as an extra unpaired T, did not lead to stalling or integration. However, a 1 or 2 base gap led to PFV integration in 20% of search events (FIG. 11B). These small gaps interrupt continuous contact of the intasome with the DNA backbone. The gaps functionally acted as “speed bumps”, stalling the PFV intasomes and allowing integration. Analysis of the reaction products by gel electrophoresis revealed that the stalling led to integration precisely at the DNA damage site. This is the first demonstration of site-specific integration by any retroviral IN. These studies show it is possible to direct PFV intasome integration site choice.

Studies of intasome dynamics with naked DNA substrates are informative, but the natural target of integration is chromatin. A nucleosome is 147 bp of nucleosome positioning sequence (NPS) DNA wrapped ˜1.7 times around an octamer of histone proteins. The octamer is comprised of two each of H2A, H2B, H3, and H4. Many transcription factors and DNA damage sensors search by continuous contact with DNA, similar to PFV intasomes. When these factors encounter a nucleosome, they take advantage of transient NPS DNA unwrapping from the histone octamer. The proteins then slide on the unwrapped portion of NPS DNA until they encounter their sequence specific binding site or DNA damage. The ability of PFV intasomes to similarly utilize transient NPS unwrapping was tested. In this case, transient unwrapping of nucleosome DNA has no effect on PFV integration site choice or efficiency. This informs the understanding of how PFV intasomes interact with targets.

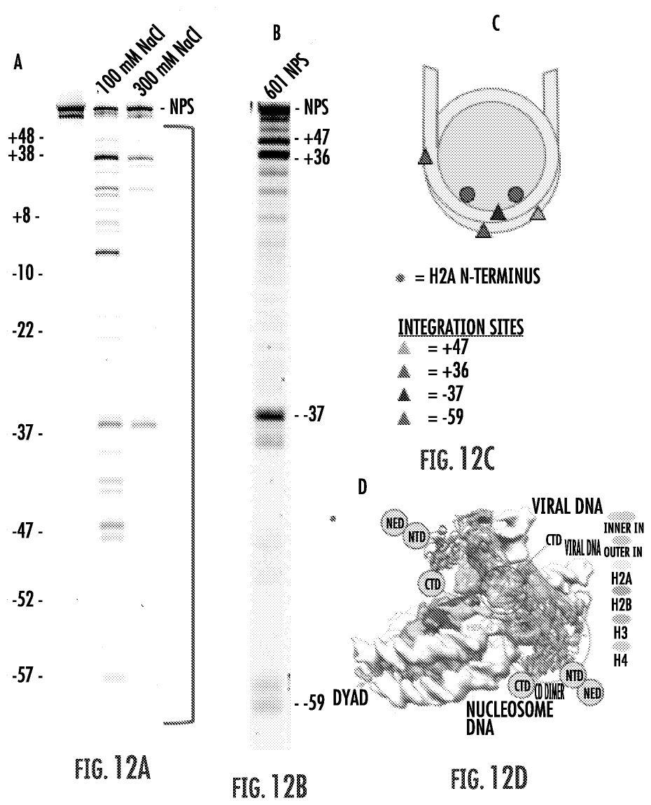

. A cryo-EM structure of a PFV intasome bound to a nucleosome showed that the CTD of an inner protomer binds to the N-terminus of H2A (FIG. 12D). This structure also indicated that PFV intasomes bind at a single symmetric site on a nucleosome, but these studies were performed at non-physiological ionic strength conditions. PFV intasome integration was assayed under physiologically relevant conditions and it was found that integration can occur at multiple sites throughout the nucleosome (FIG. 12A). This is an important finding for targeting integration in a cellular genome as it can be difficult to predict the nucleosome occupancy of the targeted site.

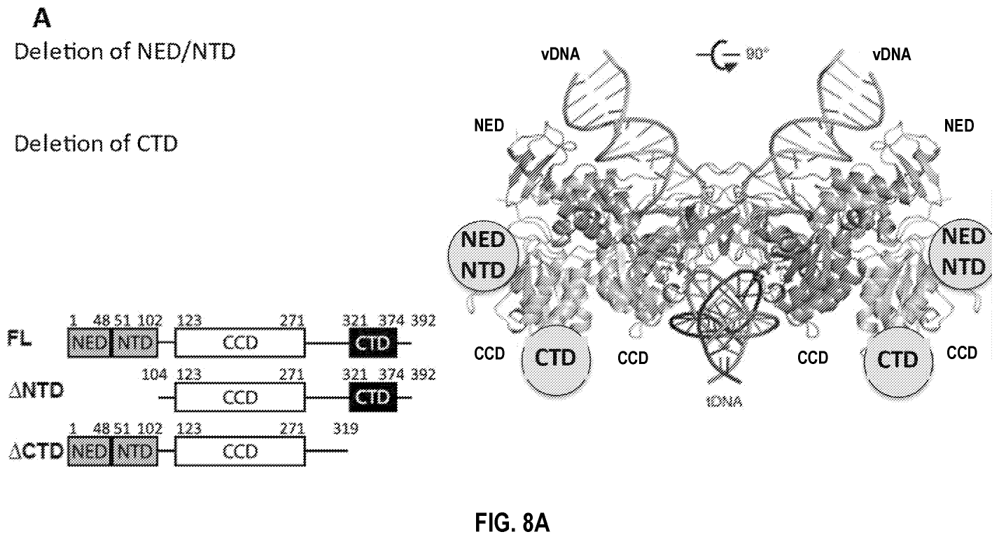

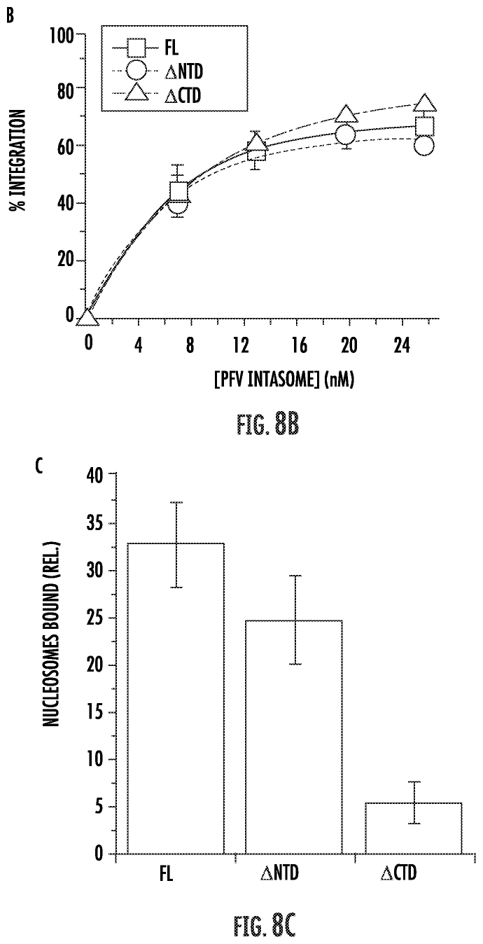

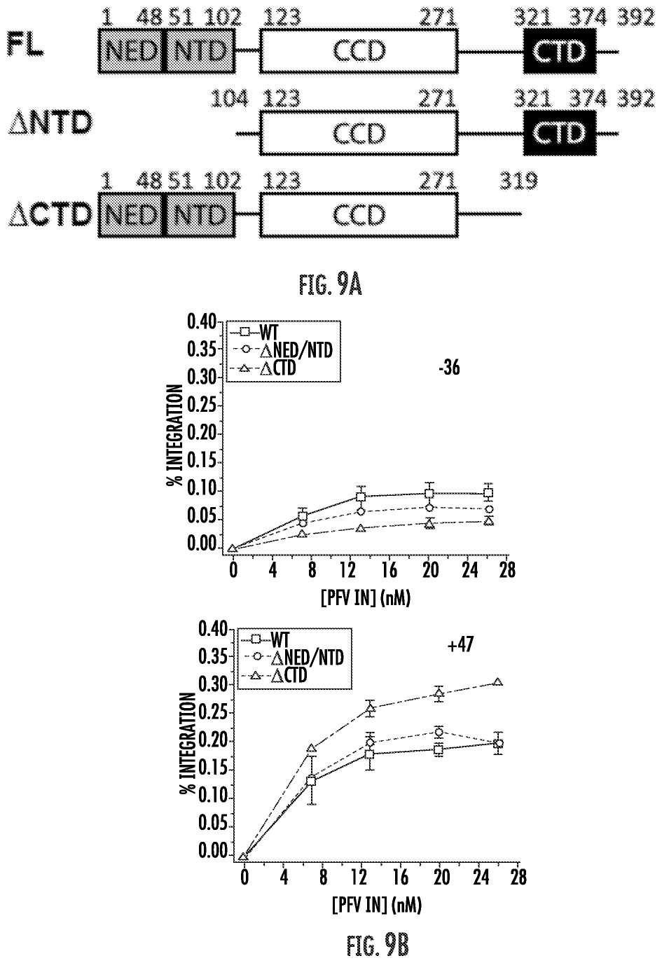

171. PFV intasomes were further tested with a highly stable synthetic nucleosome positioning sequence (NPS) termed 601. This synthetic NPS allows for specific identification of PFV integration sites around the nucleosome at physiologically relevant conditions. There were 4 major clusters of integration sites with the 601 nucleosomes (FIG. 12B). Three of these sites are near the two H2A N-termini (FIG. 12C). While one inner IN protomer bound one H2A, the other H2A can be bound by an outer IN protomer. Intasomes were compared with truncations of outer PFV IN protomers (FIG. 8A). These complexes were assayed for intasome binding to nucleosomes as well as integration efficiency and site choice. Deletion of the outer PFV IN NTDs had no effects on any of the assays. In contrast, deletion of the outer PFV IN CTDs dramatically reduced binding to nucleosomes, but increased total integration efficiency (FIGS. 8B and 8C). In addition, deletion of the outer PFV IN CTDs altered the integration site choice on the nucleosome. These data indicate that it is possible to manipulate PFV integration binding to chromatin targets and to manipulate integration site choice. While deletion of the outer PFV IN protomer CTDs reduces nucleosome binding, residual binding is mediated by several residues in the inner PFV IN protomer CTDs.

### Adding Exon Cargo to PFV Intasomes

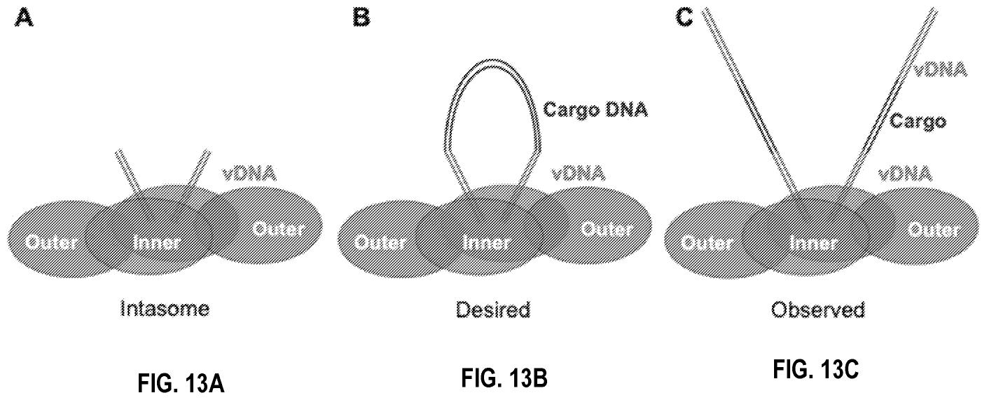

172. The goal is a purified PFV intasome with a fraction of the wild type CFTR gene, such as exon 12, a “superexon” of exons 12-27, or the entire transgene. These CFTR sequences include splice acceptor/donor and poly A signal sequences as appropriate. The CFTR sequence is flanked by vDNA. The sequence of interest is termed “cargo” DNA (FIG. 13B).

173. Currently PFV and all other retroviral intasomes are assembled with relatively short <50 bp vDNA oligomers that mimic the viral cDNA ends (FIG. 13A). The vDNA sequence is recognized by IN and unique to each retrovirus. One method to generate PFV intasomes with cargo is to assemble intasomes with a long cargo DNA and the vDNA sequence at both ends. The goal is a tetramer of PFV IN with a single long cargo DNA (FIG. 13B). However, the addition of long DNAs with vDNA sequence at each end empirically results in two DNAs in the complex (FIG. 13C). It is not technically feasible to generate PFV intasomes with cargo by this approach. Instead, the method must start with a PFV intasome assembled with oligomer vDNAs and subsequent ligation of the cargo to the distal vDNA ends.

A charged moiety at the exposed end of the vDNA oligomers inhibits PFV intasome assembly (FIG. 16B). This has proven true for a Cy5 fluorophore, a phosphate, or single stranded DNA overhangs. Efficient PFV intasome assembly requires the vDNA to be blunt and unmodified at the exposed distal end.

One approach following assembly of intasomes with blunt end vDNA is to digest with a restriction endonuclease. The cargo DNA can then be ligated to the nascent single strand DNA overhangs. However, it has been shown that 25 nM PFV intasomes aggregate within 5 min at 37° C. at salt concentrations typical for restriction endonuclease activity (110 mM NaCl). Thus, restriction endonuclease cleavage is incompatible with maintaining soluble PFV intasomes with integration activity.

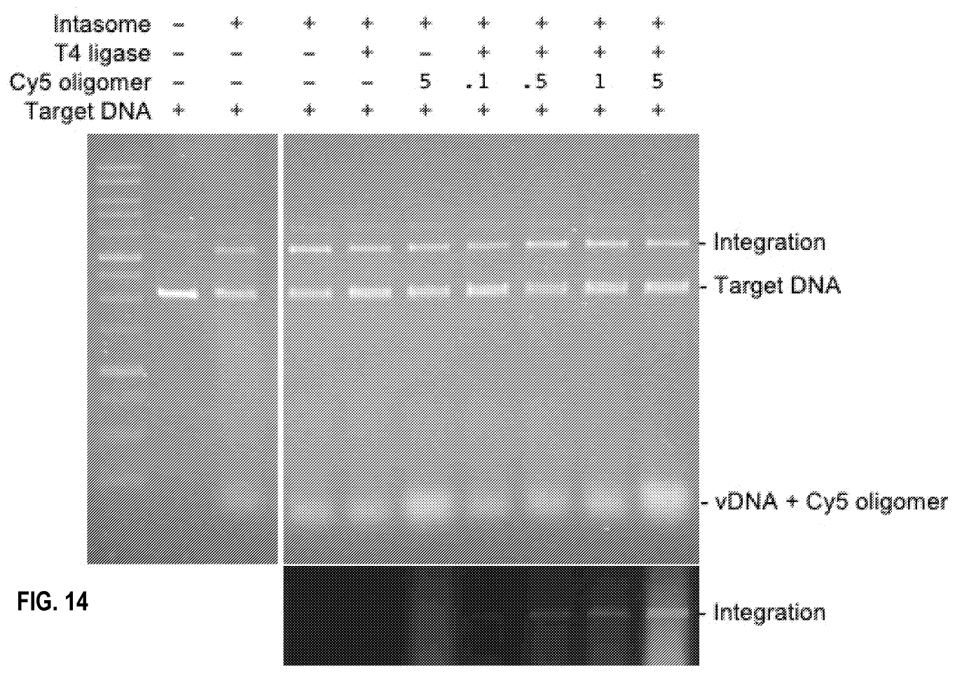

Ligation of DNA to PFV intasomes with blunt end vDNA is possible. PFV intasomes were assembled with blunt end vDNA and purified by size exclusion chromatography. The PFV intasomes were incubated at 14° C. with T4 ligase and a double strand DNA oligomer with a phosphate group at one end and a Cy5 fluorophore at the other end. The lower temperature preserves the integration activity of the intasomes, prevents intasome aggregation, and allows ligase activity. The intasomes were assayed for integration activity following ligation (FIG. 14). Integration reaction products were separated by agarose gel electrophoresis and visualized by Cy5 fluorescence. The appearance of integration products with Cy5 fluorescence indicates that the Cy5 linker DNA was successfully ligated to the intasome vDNA and the intasomes retained integration activity. This is the first demonstration of any DNA being added to purified intasomes.

### Experimental Design and Methods.

Disclosed herein are engineered synthetic PFV intasomes that carry a cargo DNA and are targeted to the CFTR locus by a ZF protein. The engineered intasomes are characterized in vitro and in cells. Biochemistry, mass photometry, and single molecule visualization techniques are used to optimize the assembly of intasomes with cargo DNA and targeting of a specific sequence. The engineered intasomes are delivered into cultured cells and assayed for site-specific integration. Develop sequence-specific targeted recombinant intasomes.

The present study generates recombinant intasomes with chimeric IN and cargo DNA. The novel intasomes are characterized for integration activity and target site selectivity in vitro

A. Demonstrate Ligation of a Cargo DNA into Purified PFV Intasomes.

179. Intasome assembly with a single long DNA cargo has not yet been reported by any laboratory. Intasome assembly is most efficient with an unmodified vDNA with blunt distal ends. A phosphorylated linker oligomer with one blunt end and one DNA overhang end can be efficiently ligated to the intasome vDNA. Subsequently a dephosphorylated cargo DNA with complementary DNA overhangs is ligated to the intasomes (FIG. 15).

Small scale assembly of PFV intasomes and ligation of linker and cargo DNA are assayed for assembly by mass photometry (FIG. 16A). Addition of a single exon 12 cargo adds >175 kDa mass, readily distinguished by single molecule mass photometry from the absence of cargo or the presence of 2 cargo DNAs. Intasomes can also be assayed for integration activity (FIG. 14). Once conditions are established for efficient assembly, larger scale intasome assemblies are purified by size exclusion chromatography and assayed for integration activity in vitro (FIG. 16B).

There are key features of this cargo DNA that minimize future risk to patients. First, there are no viral promoters or enhancers that can activate host oncogenes. Only 14 bp of the viral cDNA ends are required for IN binding and assembly. Second, there are no exogenous promoters that can activate oncogenes. The DNA cargo is either a single exon or superexon targeted to the endogenous CFTR locus. Previous studies employing HDR have demonstrated that a single exon or a superexon encoding exons 9-27 or 11-27 flanked by a splice acceptor and poly adenylation signal are able to complement CFTR mutant cells. The endogenous CFTR genomic transcription regulatory elements can drive the expression of the DNA cargo. Importantly, since the PFV intasomes are not packaged in a virus, the PFV intasomes can carry a cargo DNA large in size (e.g., 13 kb in length).

There are methods for enhancing the ligation of a single cargo, such as dilution of the reaction, altering the length of the cargo DNA, and/or addition of polyethylene glycol The use of mass photometry to quickly determine the ligating of one or two cargo DNAs.

### B. Demonstrate Sequence Specific Integration of ZF Targeted PFV Intasomes In Vitro.

Currently PFV intasomes are assembled from recombinant IN and vDNA oligomers. Compensatory point mutations direct IN to either the inner (K120E) or outer (D273K) protomers (FIG. 13A). The inner protomers must remain full length IN to bind the vDNA, form the intasome complex, and perform catalysis. The outer protomer NED, NTD, and CTD are dispensable (FIG. 8A). Deletion of the outer protomer CTDs leads to greater integration to both supercoiled plasmid DNA and 601 nucleosomes (FIG. 8B). The outer protomer CTDs are a major contributor to binding nucleosomes but not supercoiled plasmid DNA, indicating these domains bind nucleosome proteins and not NPS DNA. Deletion of the outer protomer CTDs reduces non-productive binding of PFV intasomes to cellular chromatin and can reduce off-target integration.

184. The outer protomer IN CTDs are replaced with ZF domains known to target intron of the CFTR gene (ZF_{CFTRin11}) upstream of CFTR exon 12 that encodes the G542X mutation.

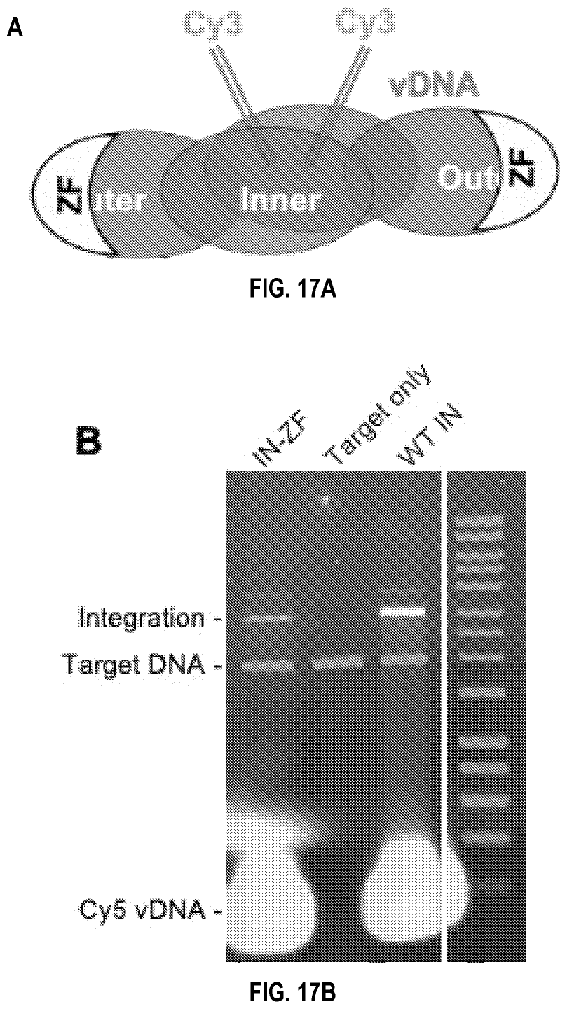

185. ZF_{CFTRin11} is engineered to PFV IN (D273K, ΔCTD) and purified. A PFV IN-ZF chimeric protein with a ZF targeting the HIV-1 LTR were purified (FIG. 17A). The protein, not assembled as an intasome, was added to vDNA and target DNA in a bulk biochemical assay of integration (FIG. 17B). Compared to wild type PFV IN, the fusion was nearly as active for integration. This result indicates that an IN-ZF chimera can assemble to enzymatically functional integration complexes. The IN-PFV IN (D273K, ΔCTD, ZF_{CFTRin11}) is assembled to intasomes with PFV IN (K120E) and fluorophore labeled vDNA. The vDNA oligomers are labeled with Cy3 or Cy5 to allow visualization of integration products in gel assays and intasome dynamics in smTIRF microscopy. The intasomes are assayed for integration to plasmid DNA with or without the ZFC_{FTRin11} binding site. The IN-ZF_{CFTRin11} intasomes are compared to PFV IN (D273K, ΔCTD) intasomes. Integration reaction products are sequenced to determine the ability of ZF_{CFTRin11} to target integration.

186. IN-ZF_{CFTRin11} intasomes are visualized by FRET and smTIRF microscopy. This method employs a 60 bp target DNA attached to a surface and with an internal Cy5 fluorophore positioned 15 bp from a ZFCFTRin11 binding site (FIG. 11A). PFV IN (ΔCTD) intasomes can slide along the target DNA without stalling or integration. However, the ZFCFTRinh can stall the intasomes and generate a FRET signal.

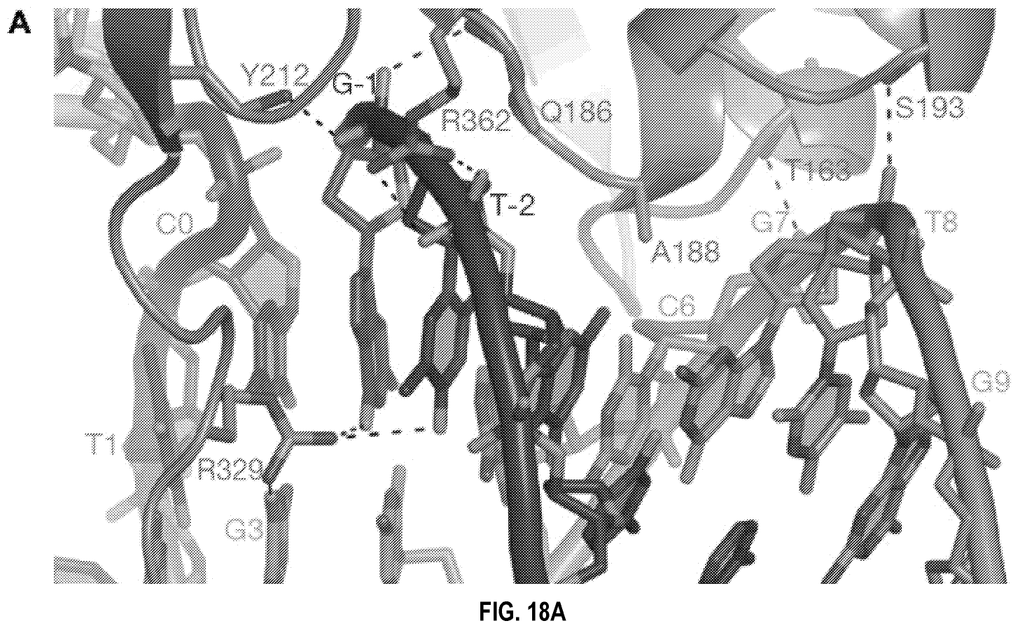

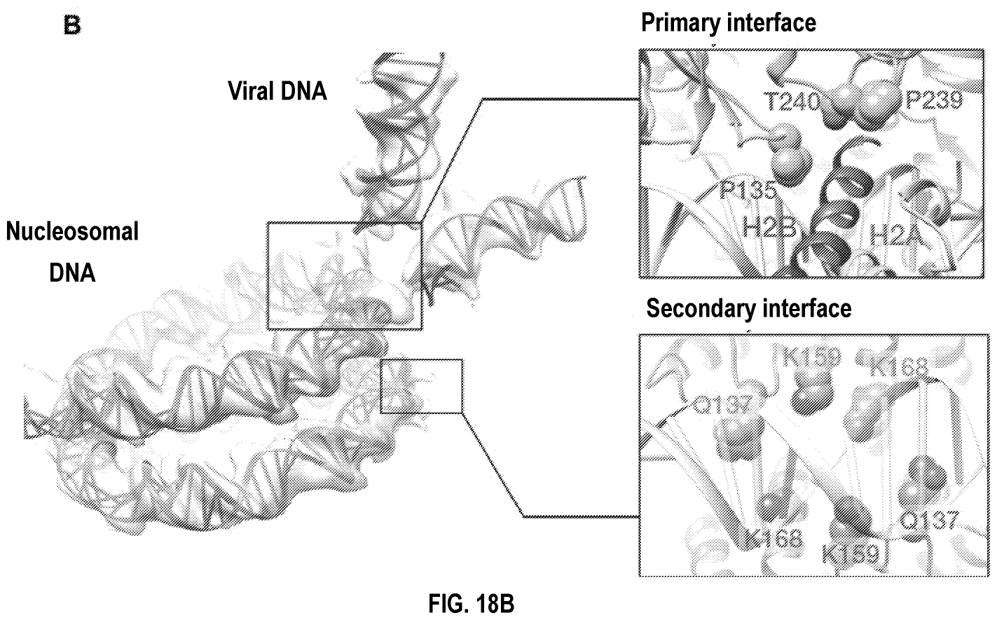

187. There is modest targeting of PFV IN-ZFCFTRin11 during the integration and search assays. PFV IN has a subtle sequence preference at the integration site. The PFV intasome structure revealed 7 amino acids of the inner protomers that form hydrogen bonds with the target DNA (FIG. 18A). These residues have not been thoroughly analyzed for their influence on target DNA binding. None of these amino acids are involved in IN catalysis (the catalytic triad is D128, D185, E221). One of these residues (R329) intercalates to the target DNA at the points of joining. Intercalation of this amino acid side chain is not required for integration activity because it is not conserved in other retroviral INs. In addition, PFV IN (R329S) retains integration activity in vitro yet reduces the sequence preference at the integration site. These results show that changing the inner protomers to PFV IN (R329S) can reduce the target preferences conferred by PFV IN. Alanine residues at several of these 7 amino acids in PFV IN (K120E) can be substituted and assembled with PFV IN-ZF_{CFTRin11} or the control PFV IN (D273K, ΔCTD). This assay can identify inner protomer mutations that abolish integration when coupled with PFV IN (D273K, ΔCTD) yet allow integration with PFV IN-ZF_{CFTRin11} to a target encoding the ZFCFTRin11 target sequence. Integration products are sequenced for confirmation of Z _{CFTRin11} targeting. Any PFV IN DNA sequence preference is abolished so that all target binding is driven by ZF_{CFTRin11}.

Limiting the targeting of PFV intasomes to the ZF_{CFTRin11} binding site also requires reduced PFV IN binding to nucleosomes. A cryo-EM structure of a PFV intasome bound to a nucleosome showed that the inner protomer has 3 residues (P135, P239, and T240) that contact H2A and H2B amino terminal tails (FIG. 18B). In addition, three residues of both the inner and outer protomer CCDs (Q137, K159, and K168) bind the second gyre of the NPS (FIG. 18B). The roles of these amino acids on binding of intasomes to nucleosomes are difficult to decipher as binding experiments were performed at ionic strength concentrations well above physiological relevance. In addition, investigation of amino acid substitutions often employed change of charge. While such charge inversion led to abolition of integration that can have been partially due to repulsion of the target DNA. Alanine substitutions of relevant residues can be employed.

PFV IN mutants are engineered at the inner and outer protomers to reduce intasome binding to target DNA and histones. The mutant intasomes are screened for integration to plasmid DNA and nucleosomes. They can also be assayed for nucleosome binding by affinity precipitation. Evaluate the Integration Profiles of PFV IN Chimeras in Cells

This study assays the ability of targeted PFV IN to integrate at specific sites in cells, delivering purified intasomes targeting the CFTR locus.

### A. Profile the Integration Sites of IN Mutants Delivered by Viral Vector Particles.

It is unlikely that retroviral vector particle delivery successfully targets integration to a single site. However, while methods for delivery of PFV intasomes to cells are developed, retroviral vector transduction is the only method for characterization of integration sites in genomes. Retroviral vectors typically deliver few integration complexes to the nucleus, but increasing the number of targets to 104 or 105 can correlate with targeting efficiencies better than random.

192. Unlike other retroviral genera, PFV integration displays little preference for any genomic feature, such as promoters (TSS+2.5 kb), active transcription units (TU), or CpG islands (Table 1). However, the viral structural protein Gag tethers the PFV integration complex to chromatin leading to minor preferences for TSS and CpG islands. The mutation PFV Gag (R540Q) abolishes these preferences indicating the PFV IN has no inherent preference for genomic features. The PFV vector particles tested herein employs the Gag (R540Q) mutation. Importantly, PFV Gag are not present in purified PFV intasomes delivered to cells in this study.

The ratio of targeted IN to genomic target sites can be manipulated by increasing the target number. This study establishes that targeted PFV intasomes are able to identify and integrate at a specific genomic site without integrating at off-target sites. Targeted integration are not as efficient as CRISPR since these PFV experiments do not recapitulate the ratio of 107 CRISPR RNPs to 2 target sites per genome; however, PFV IN targeting can be better than random and off-target integration is reduced. PFV Gag (R540Q) vector particles are engineered to express PFV IN (K120E) and mutations of the outer protomer PFV IN (D273K): truncation mutant IN ΔCTD (no targeting), IN-ZFAlu fusion targeting Alu repeats (>150,000 repeats per human genome), IN fused to an ING2-PHD domain targeting H3K4me3 (>27,000 H3K4me3 per 293T genome according to ENCODE), and IN-ZFCFTRin11 fusion targeting CFTR intron 11 (2 alleles per genome).

The PFV vector particles with chimeric IN are added to easily transduced cells such as 293T or Hela. The integration efficiency is measured by expression of a GFP reporter gene driven by an internal CMV promoter. The genomic DNA is purified, fragmented, linker adapters ligated to fragments, and integration site junctions PCR amplified with primers to the PFV genome and linkers. Integration sites are analyzed by next generation sequencing (NGS). These experiments give insight to the relative on- and off-target efficiency of modified PFV integration complexes.

### B. Deliver PFV Intasomes to Cells by Lipofection and Electroporation and Profile Integration Sites.

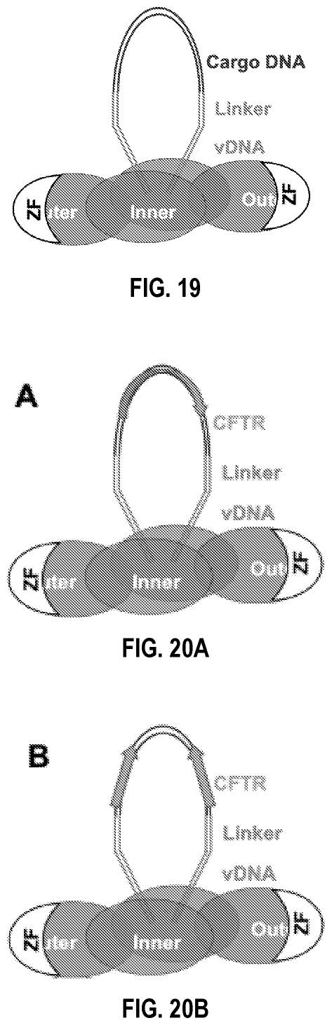

195. PFV IN-ZF_{CFTRin11} intasomes are assembled with vDNA oligomers, purified by size exclusion chromatography, and ligated to linker and cargo DNA (FIG. 19). These assemblies include the chimeric PFV INs that have a variety of targets per genome. Integration activity is confirmed by integration to a supercoiled plasmid with the matched target sequence and agarose gel electrophoresis (FIGS. 14 and 17). The cargo DNA encodes a CMV promoter driving expression of a GFP gene. Similar to CRISPR/Cas9 RNPs, the PFV intasomes are transfected to target cells by lipofection and electroporation. The first experiments focus on cells that are readily transfected without a loss of viability, such as 293T and Hela. The integration efficiency is assayed by flow cytometry of GFP expression. The integration site profile is evaluated by NGS.

196. These experiments allow the titration of the molar amount of PFV intasomes per genome. PFV intasomes with oligomeric vDNA are routinely purified to a final concentration of ˜500 nM (>10¹¹ complexes/μl). The ligation of linkers and cargo DNA can reduce the concentration of complexes. However, PFV intasomes can be concentrated with filter units. A molar excess of PFV intasomes per genome can be achieved to effectively test this technology on par with 10⁷-10⁸ CRISPR RNPs per cell. These studies show that PFV intasomes can target sequence specific sites in a cellular context.

The intasomes should not dissemble since all retroviral INs display strong binding to vDNA. In addition, to improve solubility of PFV intasome, protocatechuic acid or acetylated BSA can be added. Furthermore, aggregation was largely mediated by the outer protomer CTDs, which is not present in these modified intasomes. Moreover, an Sso7d solubility domain is added to the amino terminus of the outer PFV IN promoters to enhance intasome solubility. Sso7d has been shown to dramatically enhance the solubility of monomeric HIV-1 IN. This is the first demonstration of increasing the solubility of an intasome with the addition of Sso7d. This solubility domain has inherent DNA binding which have been abolished with the alanine substitutions of the WARE residues that mediate this activity. These alternative approaches can address any intasome solubility issues.

### C. Determine Expression of Exogenous CFTR Exons in Cells of Interest.

The engineered PFV intasomes with wild type CFTR sequence can be delivered to relevant isogenic CFTR cell lines. Pulmonary epithelial cells were engineered with CRISPR methods to be homozygous for CFTR mutations F508del, G542X, and W1282X. The first assay focuses on the G542X mutant for three reasons. First, it is the second most prevalent mutation in patients. Second, it is not treated with the current CF drug regimen. Third, the PFV IN-ZFCFTRint complementation can be readily determined. Successful integration and rescue of CFTR protein expression can be detected by Western analysis or flow cytometry. The matched parental cell line expresses CFTR and serves as a positive control. The untreated G542X cell line does not express CFTR and serves as a negative control. PFV IN-ZFCFTRinh intasomes are added to G542X cells followed by analysis of CFTR protein expression. The integration sites are assayed by NGS to confirm appropriate on-target insertion and lack of off-target integration.

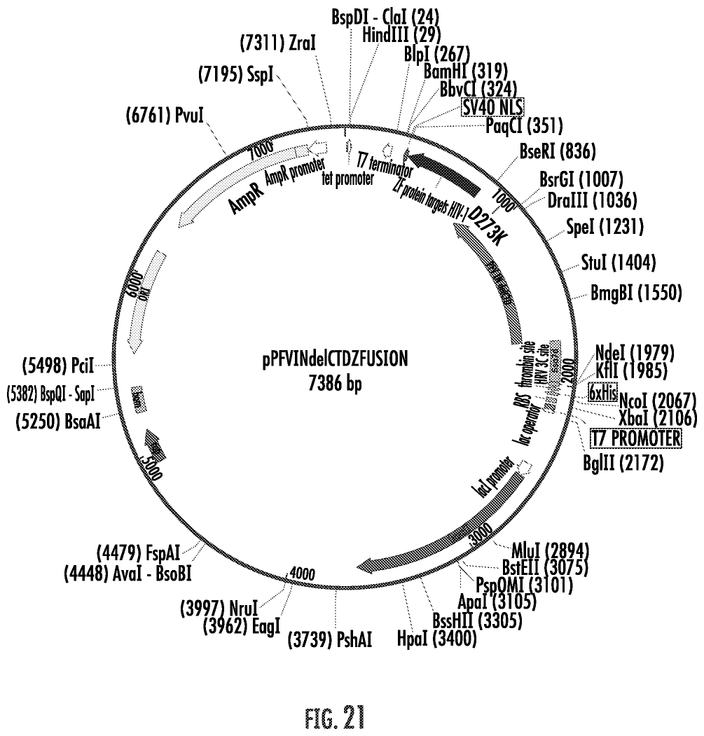

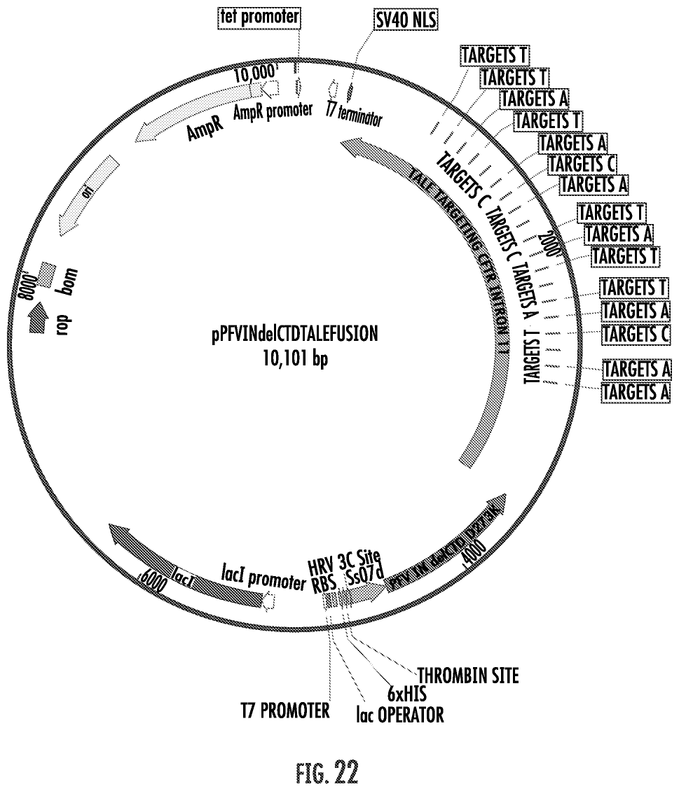

PFV intasomes are symmetric and do not integrate with a preference of in-frame or in reverse orientation within the CFTR locus. Two iterations of the cargo DNA are assayed (FIG. 20). The first has only a single copy of the CFTR sequence. The second cargo DNA has two copies of the CFTR sequence in reverse orientation to each other. Signal sequences such as splice acceptor/donor and poly A are included as appropriate.

. The PFV IN is expressed in E. coli bacteria and the protein is purified. Th synthetic viral DNA oligomers are added to the purified PFV IN. The viral DNA and PFV IN are placed in dialysis tubing and the salt concentration is slowly changed. This process allows the complexes to form. Then the complexes are purified by size exclusion chromatography. After the integration complexes are purified, the linker DNA is ligated. And after that linker ligation, the cargo DNA is ligated.

Integrase binds the vDNA in a sequence specific, non-covalent binding. The viral DNA sequence is unique to each retrovirus so that the viral DNA oligomers and IN must be from the same retrovirus. IN recognizes ˜14 bp of the viral DNA termini.

Viral DNA of the intasome is generated by single strand DNA oligomer oKEY616 annealed to single strand DNA oligomer oKEY75. These oligomers are not modified.

OKEY616:
(SEQ ID NO: 22)
5′-ATTGTCATGGAATTTTGTATATTGAGTGGCGCCCGAACAG-3′
OKEY675:
(SEQ ID NO: 23)
5′-CTGTTCGGGCGCCACTCAATATACAAAATTCCATGACA-3′

Linker oligomers have a blunt end to ligate to the viral DNA blunt end and a BamHI overhang end to ligate to cargo DNA. These oligomers have 5′ phosphate groups which are required for ligation.

OKEY1000:
(SEQ ID NO: 24)
5′ P-gtcgacCCAATTGGGCGCGCCTTAATTAACATATGg 3′
OKEY999:
(SEQ ID NO: 25)
5′ P-gatccCATATGTTAATTAAGGCGCGCCCAATTGGgtcgac 3′

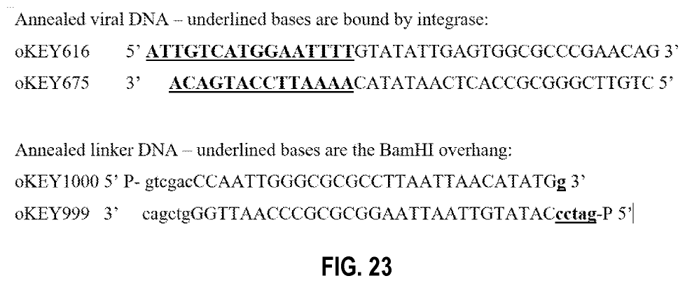

The annealed viral DNA and the annealed linker DNA are shown in FIG. 23.

### Materials and Methods.

DNA substrates. A DNA oligonucleotide with an internal amino-modified thymine (T*) at the fourth base from the 5′ end (5′-CTGT*AGAATCCCGGTGCCGAGGCCGCT-3′ (SEQ ID NO: 26) Integrated DNA Technologies) was labeled with Cy5-NHS ester (GE Healthcare). The labeled oligonucleotide was purified by reverse-phase HPLC with a Poroshell 120 EC-C18 column (Agilent Technologies). The 147 bp 601 NPS was amplified from pDrive-601 NPS with the Cy5 labeled oligonucleotide and DNA oligonucleotide 5′-ACAGGATGTATATATCTGACACGTGCCTGGA-3′ (SEQ ID NO: 27). The resulting Cy5 labeled 601 NPS DNA was purified by ion-exchange HPLC with a Gen-Pak Fax column (Waters).

205. PFV vDNA substrates were annealed DNA oligonucleotides oKEY616 5′-ATTGTCATGGAATTTTGTATATTGAGTGGCGCCCGAACAG-3′ (SEQ ID NO: 22) and oKEY675 5′-CTGTTCGGGCGCCACTCAATATACAAAATTCCATGACA-3′ (SEQ ID NO: 23) (Integrated DNA Technologies). When vDNA was modified with Cy5 or biotin, the moiety was at the 5′ end of oKEY675.

Nucleosomes. Recombinant human histones H2A or H2A (K119C), H2B, H3, and H4 were expressed and purified. Purified histone H2A (K119C) was labeled with Cy3-maleimide (GE Healthcare). Cy3-H2A allowed visualization of fluorescent histone octamers by gel analysis and FRET confirmation of predicted Cy5-NPS positioning. Histone octamers were refolded at equimolar histone concentrations and purified by Superose 12 10/300 gel filtration chromatography (GE Healthcare or Lumiprobe). Nucleosomes were reconstituted with 147 bp 601 DNA and histone octamer by double dialysis. The products were separated by sucrose gradient velocity centrifugation. Gradient fractions were analyzed by native PAGE and imaged using a Typhoon 9410 variable mode fluorescent imager (GE Healthcare). Fractions with fluorescent NPS DNA bound by nucleosomes were combined, concentrated with Amicon Ultra centrifugal filters (EMD Millipore), and stored at 4° C. All experiments were performed with at least two independent nucleosome preparations, derived from independent histone octamer refoldings. Nucleosomes were treated with trypsin (Sigma-Aldrich) at an enzyme-to-substrate ratio of 1:120 w/w for 3 h at ambient temperature. Digestion was terminated by the addition of tenfold molar excess soybean trypsin inhibitor (Sigma-Aldrich). To confirm the deletion of histone tails, nucleosomes were labeled on free amino groups by incubating with twofold molar excess Cy5 NHS ester (Lumiprobe) in 10 mM Tris-HCl (pH 7.5), 25 mM NaCl for 1 h at ambient temperature and analysed by PAGE.

SmFRET imaging was done on a home-built inverted fluorescence microscope (Olympus). Prism-based total internal reflection (TIRF) of a green (532 nm) or a red (635 nm) laser was used to excite fluorophores attached to a surface of a flow channel. The fluorescence from individual fluorophores was collected through a 60X water immersion objective (Olympus) and directed onto an emCCD camera (Princeton Instruments) after magnifying 1.6× times and separating Cy3, Cy5 emissions using a Dual View device (Photometrics).

208. The quartz surfaces of the flow channels were passivated with a 1:20 ratio of biotin-PEG and methoxy-PEG (5000 MW, Layson Bio, Inc). Biotin-PEG was used to immobilize target DNAs via biotin-neutravidin-biotin interactions at ˜0.2 molecules/μm² surface density. The methoxy-PEG brush minimizes the surface interactions of biomolecules. The imaging buffer (Buffer-I) for all the experiments consisted of 30 mM Bis-tris propane, pH 7.5, 110 mM NaCl, 2 mM MgSO₄, 4 μM ZnCl₂, 0.1 mM DTT, 0.2 mg/mL BSA, 0.02% IGPEPAL. Buffer-I also included a cocktail of saturated (˜2 mM) Trolox and an oxygen scavenging system (OSS) to minimize photo-blinking and photobleaching of fluorophores, respectively. The OSS consisted of 25 mM protocatechuic acid (PCA) and 20 nM protocatechuate dioxygenase (PCD). All the experiments were performed at 24±1.0 C.

The imaging for target capture was done at 100 ms time resolution to detect transient events. First, single-molecule movies were initiated by exciting Cy5 on the target DNAs with the 635 nm red laser at ˜2 mW. After 20 s, the excitation was switched to the 532 nm green laser maintained at ˜6 mW. 10 s after the green laser exposure; Buffer-I containing 5 nM Cy3 tagged PFV intasomes was infused in real-time into the flow channels. Data recording proceeded under continuous green laser excitation for 2.5 min from the injection.

PFV integration. PFV intasomes were assembled and purified. All experiments were performed with at least two independent intasome purifications. PFV intasomes with truncation mutants at the outer IN positions were assembled using equimolar concentrations of PFV IN (K120E) and truncation mutants PFV IN (D273K, ANEDANTD) or PFV IN (D273K, ΔCTD). Unless otherwise noted, integration reactions contained 10 mM Bis-tris propane-HCl (pH 7.5), 110 mM NaCl, 5 mM MgSO⁴, 4 μM ZnCl₂, 10 mM DTT, the indicated concentration PFV intasomes, and 15 ng NPS DNA in a final volume of 15 μl. Time course reactions included 13 nM PFV intasomes. Reactions were incubated at 37° C. for 5 min and stopped with 0.1 volumes stop solution (5% SDS, 10 mg/ml proteinase K) and 0.05 volumes 500 mM EDTA. Reactions were further incubated at 55° C. for 1 h. Products were separated by native or denaturing PAGE and scanned with a Typhoon 9410 variable mode fluorescent imager (GE Healthcare) or Sapphire Biomolecular Imager (Azure Biosystems). Molecular weights were calculated by fitting standards (GeneScan 120 LIZ Size Standard, Thermo Fisher Scientific) to an exponential curve. The standard curve was then used to determine the molecular weight of each band (+3 nucleotides [nt]) depending on pixel position. To calculate the relative intensity of each band or integration cluster, the total fluorescence intensity of each lane was quantified (BioNumerics 7.6, Applied Maths). This software converts each gel lane to a densitometric curve in which each band appears as a peak. Least square filtering was used to remove background. The total fluorescence intensity of each lane is the sum of all bands in a lane. The integrated area under each band peak was then calculated. This value was divided by the total fluorescence of the lane and multiplied by 100 to yield the percent of total fluorescent signal (OriginPro 9.1, OriginLab). The percentage of the total fluorescence in the lane is referred to as integration efficiency. Individual peaks in a cluster of bands could not be integrated individually as a result of overlapping pixel densities. The data are presented as averages±standard deviation (sd) of at least three independent experiments. p values were determined using a two-tailed t-test at a 95% confidence interval. Total integration efficiency was determined by subtracting the fraction of unreacted NPS from the total fluorescent signal in each lane.

PFV intasomes binding to nucleosomes. Binding to nucleosomes was assayed by affinity precipitation of biotinylated intasomes with streptavidin-coated beads. PFV intasomes were assembled with biotinylated vDNA. In total, 10 μg of PFV intasomes was added to 10 μg of nucleosomes (EpiCypher) in wash buffer (50 mM HEPES (pH 7.5), 110 mM or 300 mM NaCl, 10% glycerol, 1 mM DTT, 0.1% Tween, and 1 μg/ml BSA) in a final volume of 350 μl. Samples were incubated on ice for 20 min followed by room temperature for 30 min. In total, 70 μl of streptavidin-conjugated magnetic beads (Dynabeads M-280 streptavidin, Invitrogen) was washed with three volumes of wash buffer and resuspended in 17.5 μl of wash buffer. The magnetic beads were added to 333.5 μl of each sample. The remaining 17.5 μl of intasomes with nucleosomes was saved for gel analysis as a 5% input control. Samples with magnetic beads were slowly rotated at room temperature for 1 h. The beads were then washed with three volumes of wash buffer. Beads were resuspended in phosphate-buffered saline (PBS, Sigma-Aldrich) and SDS-PAGE loading dye, boiled for 10 min, and analyzed by SDS-PAGE. Gels were stained with Coomassie Brilliant Blue R-250 (Amresco) and imaged (Epson scanner or Azure Sapphire Biomolecular Imager). Coomassie stained protein bands were quantitated (ImageJ). The total signal in each lane was determined, excluding the overlapping bands of streptavidin and H4. The histone bands were calculated as the percentage of the total signal in each lane. Averages and standard deviations were derived from at least three experiments with two independent PFV intasome and nucleosome preparations.

1. An engineered retroviral integration complex comprising an engineered prototype foamy virus (PFV) integrase (IN), two or more non-naturally occurring flanking nucleic acid sequences, and a cargo nucleic acid sequence.

2. The engineered retroviral integration complex of claim 1, wherein the PFV integrase comprises at least one inner PFV IN protomer and at least one outer PFV IN protomers.

3. The engineered retroviral integration complex of claim 1, wherein the PFV integrase comprises two inner PFV IN protomers and two outer PFV IN protomers.

4. The engineered retroviral integration complex of claim 2, wherein the outer PFV IN protomer comprises a nucleic acid binding domain.

5. The engineered retroviral integration complex of claim 1, wherein the flanking nucleic acid sequences are derived from a virus.

6. The engineered retroviral integration complex of claim 5, wherein the flanking viral nucleic acid sequence is DNA.

7. The engineered retroviral integration complex of claim 4, wherein a carboxyl terminus domain (CTD) region of the outer PFV IN protomer is replaced with the nucleic acid binding domain.

8. The engineered retroviral integration complex of claim 4, wherein the nucleic acid binding domain is a transcription activator-like effector (TALE), a zinc finger (ZF) domain, or a Cas9/gRNA complex.

9. The engineered retroviral integration complex of claim 4, wherein the nucleic acid binding domain targets a human gene.

10. The engineered retroviral integration complex of claim 9, wherein the human gene is a cystic fibrosis transmembrane conductance regulator (CFTR) gene, human Alu repeats, or a portion thereof.

11. The engineered retroviral integration complex of claim 8, wherein the ZF domain does not require dimerization.

12. The engineered retroviral integration complex of claim 8, wherein the TALE comprises a sequence at least 80% identity to SEQ ID NO: 28 or a fragment thereof.

13. The engineered retroviral integration complex of claim 2, wherein the amino terminus of the outer PFV IN protomer is linked to a Sso7d solubility domain.

14. (canceled)

15. (canceled)

16. (canceled)

17. (canceled)

18. (canceled)

19. (canceled)

20. The engineered retroviral integration complex of claim 1, wherein the flanking nucleic acid sequences comprise two blunt ends, one sticky end, or two sticky ends.

21. (canceled)

22. (canceled)

23. The engineered retroviral integration complex of claim 1, wherein the cargo nucleic acid sequence is flanked by the flanking nucleic acid sequences.

24. The engineered retroviral integration complex of claim 23, wherein the cargo nucleic acid sequence is linked to at least one flanking nucleic acid sequence through a polynucleotide linker sequence.

25. (canceled)

26. The engineered retroviral integration complex of claim 24, wherein the polynucleotide linker sequence comprises a blunt end linked to the flanking nucleic acid sequence and a sticky end linked to the cargo nucleic acid sequence.

27. (canceled)

28. (canceled)

29. (canceled)

30. (canceled)

31. (canceled)

32. (canceled)

33. An expression vector comprising a polynucleotide sequence encoding the engineered retroviral integration complex of claim 1.

34. The expression vector of claim 33, wherein the polynucleotide sequence is at least 80% identical to SEQ ID NO: 21 or 34.

35. An engineered cell comprising the engineered retroviral integration complex of claim 1.

36. A method of expressing an exogenous nucleic acid in a subject in need thereof, comprising administering to the subject an engineered retroviral integration complex comprising an engineered prototype foamy virus (PFV) integrase (IN), two or more non-naturally occurring flanking nucleic acid sequences, and a cargo nucleic acid sequence, wherein the cargo nucleic acid is expressed in the subject.

37-118. (canceled)


SEQUENCE LISTING (34 sequences)

SEQ ID NO: 1 - DNA, 6000 bp, Homo sapiens
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaa

SEQ ID NO: 2 - DNA, 5997 bp, Homo sapiens
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatcggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaa

SEQ ID NO: 3 - DNA, 6000 bp, Homo sapiens
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttctttgagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaa

SEQ ID NO: 4 - DNA, 6000 bp, Homo sapiens
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtgaaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaa

SEQ ID NO: 5 - DNA, 1179 bp, Artificial Sequence
  Synthetic Construct
tgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtagtggtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaatga

SEQ ID NO: 6 - protein, 392 aa, Artificial Sequence
  Synthetic Construct
CNTKKPNLDAELDQLLQGHYIKGYPKQYTYFLEDGKVKVSRPEGVKIIPPQSDRQKIVLQAHNLAHTGREATLLKIANLYWWPNMRKDVVKQLGRCQQCLITNASNKASGPILRPDRPQKPFDKFFIDYIGPLPPSQGYLYVLVVVDGMTGFTWLYPTKAPSTSATVKSLNVLTSIAIPKVIHSDQGAAFTSSTFAEWAKERGIHLEFSTPYHPQSSGKVERKNSDIKRLLTKLLVGRPTKWYDLLPVVQLALNNTYSPVLKYTPHQLLFGIDSNTPFANQDTLDLTREEELSLLQEIRTSLYHPSTPPASSRSWSPVVGQLVQERVARPASLRPRWHKPSTVLKVLNPRTVVILDHLGNNRTVSIDNLKPTSHQNGTTNDTATMDHLEKNE

SEQ ID NO: 7 - DNA, 1179 bp, Artificial Sequence
  Synthetic Construct
tgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaagaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtagtggtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaatga

SEQ ID NO: 8 - protein, 392 aa, Artificial Sequence; identical to SEQ ID NO: 6
  Synthetic Construct

SEQ ID NO: 9 - DNA, 1179 bp, Artificial Sequence
  Synthetic Construct
tgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaagaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtagtggtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtataaaatcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaatga

SEQ ID NO: 10 - protein, 392 aa, Artificial Sequence; identical to SEQ ID NO: 6
  Synthetic Construct

SEQ ID NO: 11 - DNA, 957 bp, Artificial Sequence
  Synthetic Construct
tgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtagtggtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtataaaatcaaatactccatttgcaaatcaagatacacttgacttgaccagagaagaagaactttctcttttacaggaaattcgtacttctttataccatccatccacccctccagcctcctctcgttcctggtctcctgttgtt

SEQ ID NO: 12 - protein, 319 aa, Artificial Sequence
  Synthetic Construct
CNTKKPNLDAELDQLLQGHYIKGYPKQYTYFLEDGKVKVSRPEGVKIIPPQSDRQKIVLQAHNLAHTGREATLLKIANLYWWPNMRKDVVKQLGRCQQCLITNASNKASGPILRPDRPQKPFDKFFIDYIGPLPPSQGYLYVLVVVDGMTGFTWLYPTKAPSTSATVKSLNVLTSIAIPKVIHSDQGAAFTSSTFAEWAKERGIHLEFSTPYHPQSSGKVERKNSDIKRLLTKLLVGRPTKWYDLLPVVQLALNNTYSPVLKYTPHQLLFGIKSNTPFANQDTLDLTREEELSLLQEIRTSLYHPSTPPASSRSWSPVV

SEQ ID NO: 13 - DNA, 189 bp, Artificial Sequence
  Synthetic Construct
gcgaccgttaaattcaaatacaaaggcgaagaaaaagaagtggacatctcaaaaatcaaaaaagtcgcgcgtgtgggtaaaatgattagctttacctatgatgaaggcggtggcaaaacgggtgaaggcgcggtttctgaaaaagacgccccgaaagaactgctgcagatgctggaaaaacagaaaaaa

SEQ ID NO: 14 - protein, 63 aa, Artificial Sequence
  Synthetic Construct
ATVKFKYKGEEKEVDISKIKKVARVGKMISFTYDEGGGKTGEGAVSEKDAPKELLQMLEKQKK

SEQ ID NO: 15 - DNA, 436 bp, Artificial Sequence
  Synthetic Construct
cgccgctatggctgagaggcccttccagtgtcgaatctgcatgcgtaacttcagtcgctccgacgacctgtccaagcacatccgcacccacaccggcgagaagccttttgcctgtgacatttgtgggaggaaatttgccgaccgctcccacctggcccgccataccaagatacacacgggcagccaaaagcccttccagtgtcgaatctgcatgcgtaacttcagtcgctccgacaacctggcccgccacatccgcacccacaccggcgagaagccttttgcctgtgacatttgtgggaggaaatttgcctggcgcggcgaccgcgtgaagcataccaagatacacacgggcgagaagcccttccagtgtcgaatctgcatgcgtaagtttgcccagtccggcgacctgacccgccataccaagatacacctgcgg

SEQ ID NO: 16 - protein, 145 aa, Artificial Sequence
  Synthetic Construct
AAMAERPFQCRICMRNFSRSDDLSKHIRTHTGEKPFACDICGRKFADRSHLARHTKIHTGSQKPFQCRICMRNFSRSDNLARHIRTHTGEKPFACDICGRKFAWRGDRVKHTKIHTGEKPFQCRICMRKFAQSGDLTRHTKIHLR

SEQ ID NO: 17 - DNA, 350 bp, Artificial Sequence
  Synthetic Construct
gccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgcccaccgctccacccgcaaccgccataccaagatacacacgggatctcagaagcccttccagtgtcgaatctgcatgcgtaacttcagtcagtccggcgacctgacccgccacatccgcacccacaccggcgagaagccttttgcctgtgacatttgtgggaggaaatttgcccagcaggccaacctgggcaagcataccaagatacacctgcg

SEQ ID NO: 18 - protein, 117 aa, Artificial Sequence
  Synthetic Construct
AAMAERPFQCRICMRNFSQSSDLSRHIRTHTGEKPFACDICGRKFAHRSTRNRHTKIHTGSQKPFQCRICMRNFSQSGDLTRHIRTHTGEKPFACDICGRKFAQQANLGKHTKIHLR

SEQ ID NO: 19 - DNA, 4594 bp, Artificial Sequence
  Synthetic Construct
ggatccagattgagcatactaaaagtgactctctaattttctatttttggtaataggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttagatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttagatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaaaacttgtttattgcagcttataatggttacaaataaagcaatagcatcacaaatttcacaaataaagcatttttttcactgcattctagttgtggtttgtccaaactcatcaatgtatctta

SEQ ID NO: 20 - DNA, 4472 bp, Artificial Sequence
  Synthetic Construct
ggatccagattgagcatactaaaagtgactctctaattttctatttttggtaataggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttagatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttagatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaa

SEQ ID NO: 21 - DNA, 7386 bp, Artificial Sequence
  Synthetic Construct
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatccggatatagttcctcctttcagcaaaaaacccctcaagacccgtttagaggccccaaggggttatgctagttattgctcagcggtggcagcagccaactcagcttcctttcgggctttgttagcagccggatcctcagccaccgaccttccgcttcttctttggcccccgcaggtgtatcttggtatggcgggtcaggtcgccggactgggcaaacttacgcatgcagattcgacactggaagggcttctcgcccgtgtgtatcttggtatgcttcacgcggtcgccgcgccaggcaaatttcctcccacaaatgtcacaggcaaaaggcttctcgccggtgtgggtgcggatgtggcgggccaggttgtcggagcgactgaagttacgcatgcagattcgacactggaagggcttttggctgcccgtgtgtatcttggtatggcgggccaggtgggagcggtcggcaaatttcctcccacaaatgtcacaggcaaaaggcttctcgccggtgtgggtgcggatgtgcttggacaggtcgtcggagcgactgaagttacgcatgcagattcgacactggaagggcctctcagccatagcggcgggtacaacaacaggagaccaggaacgagaggaggctggaggggtggatggatggtataaagaagtacgaatttcctgtaaaagagaaagttcttcttctctggtcaagtcaagtgtatcttgatttgcaaatggagtatttgattttataccaaataagagttgatgtggagtatattttaatacagggctataggtgttgtttaaagcaagttgtacaacaggcaataggtcataccactttgtgggtcttcctactagcagtttagttaaaagtcgttttatatcactatttttcctttccaccttaccactactttgggggtgataaggagtactgaattccaaatgtatacctctttcctttgcccattcagcaaaggttgaagaagtgaatgctgcaccttgatcagagtgaatcacctttggaattgcaatactagtgagtacattgagagatttaacagttgcgctagtagaaggagccttagtggggtataaccaagtgaatcctgtcattccatcaacaactactaatacatataggtatccctgtgaaggtggcaaaggtccaatatagtcaataaagaatttatcaaaaggtttttgaggcctatctggtcttagaataggaccagaggctttgttggaagcatttgtgattaaacactgttgacagcgtcctagttgtttaaccacatcctttctcatatttggccaccaataaaggttggcaatttttaaaagagtggcttcacgtccggtgtgagccaaattgtgggcttgaagcacaattttttgtctgtctgactgagggggaataattttaaccccttcaggtctggaaacttttactttgccatcttctaaaaaatatgtatattgtttgggatatccttttatataatgaccctgtaataattgatccaactctgcatccaggtttggttttttggtattacaaccaccgccaccgccaccgccaccgccaccgccttttttctgtttttccagcatctgcagcagttctttcggggcgtctttttcagaaaccgcgccttcacccgttttgccaccgccttcatcataggtaaagctaatcattttacccacacgcgcgacttttttgatttttgagatgtccacttctttttcttcgcctttgtatttgaatttaacggtcgccatatggggtccctgaaagaggacttcaaggctatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 22 - DNA, 40 bp, Artificial Sequence
  Synthetic Construct
attgtcatggaattttgtatattgagtggcgcccgaacag

SEQ ID NO: 23 - DNA, 38 bp, Artificial Sequence
  Synthetic Construct
ctgttcgggcgccactcaatatacaaaattccatgaca

SEQ ID NO: 24 - DNA, 36 bp, Artificial Sequence
  Synthetic Construct
gtcgacccaattgggcgcgccttaattaacatatgg

SEQ ID NO: 25 - DNA, 40 bp, Artificial Sequence
  Synthetic Construct
gatcccatatgttaattaaggcgcgcccaattgggtcgac

SEQ ID NO: 26 - DNA, 27 bp, Artificial Sequence
  Synthetic Construct
ctgtagaatcccggtgccgaggccgct

SEQ ID NO: 27 - DNA, 31 bp, Artificial Sequence
  Synthetic Construct
acaggatgtatatatctgacacgtgcctgga

SEQ ID NO: 28 - protein, 1052 aa, Artificial Sequence
  Synthetic Construct
SRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGRLTPEQVVAIASNGGGRPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRASAS

SEQ ID NO: 29 - DNA, 3156 bp, Artificial Sequence
  Synthetic Construct
tcgcggacccggctcccttccccacccgcacccagcccagcgttttcggccgactcgttctcagacctgcttaggcagttcgacccctcactgtttaacacatcgttgttcgactcccttcctccgtttggggcgcaccatacggaggcggccaccggggagtgggatgaggtgcagtcgggattgagagctgcggatgcaccacccccaaccatgcgggtggccgtcaccgctgcccgaccgccgagggcgaagcccgcaccaaggcggagggcagcgcaaccgtccgacgcaagccccgcagcgcaagtagatttgagaactttgggatattcacagcagcagcaggaaaagatcaagcccaaagtgaggtcgacagtcgcgcagcatcacgaagcgctggtgggtcatgggtttacacatgcccacatcgtagccttgtcgcagcaccctgcagcccttggcacggtcgccgtcaagtaccaggacatgattgcggcgttgccggaagccacacatgaggcgatcgtcggtgtggggaaacagtggagcggagcccgagcgcttgaggccctgttgacggtcgcgggagagctgagagggcctccccttcagctggacacgggccagttgctgaagatcgcgaagcggggaggagtcacggcggtcgaggcggtgcacgcgtggcgcaatgcgctcacgggagcacccctcaacctgaccccagagcaggtcgtggcaatcgcctccaacattggcgggaaacaggcactcgagactgtccagcgcctgcttcccgtgctgtgccaagcgcacggactgaccccagagcaggtcgtggcaatcgcctccaacattggcgggaaacaggcactcgagactgtccagcgcctgcttcccgtgctgtgccaagcgcacggactgaccccagagcaggtcgtggccattgcctcgaatggagggggcaaacaggcgttggaaaccgtacaacgattgctgccggtgctgtgccaagcgcacggcttgaccccagagcaggtcgtggcgatcgcaagccacgacggaggaaagcaagccttggaaacagtacagaggctgttgcctgtgctgtgccaagcgcacgggctgaccccagagcaggtcgtggcaatcgcctccaacattggcgggaaacaggcactcgagactgtccagcgcctgcttcccgtgctgtgccaagcgcacggactgaccccagagcaggtcgtggccattgcctcgaatggagggggcaaacaggcgttggaaaccgtacaacgattgctgccggtgctgtgccaagcgcacggcctgaccccagagcaggtcgtggcaatcgcctccaacattggcgggaaacaggcactcgagactgtccagcgcctgcttcccgtgctgtgccaagcgcacggactgaccccagagcaggtcgtggccattgcctcgaatggagggggcaaacaggcgttggaaaccgtacaacgattgctgccggtgctgtgccaagcgcacggcctgaccccagagcaggtcgtggcaatcgcctccaacattggcgggaaacaggcactcgagactgtccagcgcctgcttcccgtgctgtgccaagcgcacggactgaccccagagcaggtcgtggccattgcctcgaatggagggggcaaacaggcgttggaaaccgtacaacgattgctgccggtgctgtgccaagcgcacggcttgaccccagagcaggtcgtggcgatcgcaagccacgacggaggaaagcaagccttggaaacagtacagaggctgttgcctgtgctgtgccaagcgcacgggctgaccccagagcaggtcgtggcaatcgcctccaacattggcgggaaacaggcactcgagactgtccagcgcctgcttcccgtgctgtgccaagcgcacggattgaccccagagcaggtcgtggcgatcgcaagccacgacggaggaaagcaagccttggaaacagtacagaggctgttgcctgtgctgtgccaagcgcacgggctgaccccagagcaggtcgtggcaatcgcctccaacattggcgggaaacaggcactcgagactgtccagcgcctgcttcccgtgctgtgccaagcgcacggattgaccccagagcaggtcgtggcgatcgcaagccacgacggaggaaagcaagccttggaaacagtacagaggctgttgcctgtgctgtgccaagcgcacgggctgaccccagagcaggtcgtggccattgcctcgaatggagggggcaaacaggcgttggaaaccgtacaacgattgctgccggtgctgtgccaagcgcacggcctgaccccagagcaggtcgtggcaatcgcctccaacattggcgggaaacaggcactcgagactgtccagcgcctgcttcccgtgctgtgccaagcgcacggactgaccccagagcaggtcgtggccattgcctcgaatggagggggcaaacaggcgttggaaaccgtacaacgattgctgccggtgctgtgccaagcgcacggccgactcacgcctgagcaggtagtggctattgcatccaatggcgggggcagacccgcactggagtcaatcgtggcccagctttcgaggccggaccccgcgctggccgcactcactaatgatcatcttgtagcgctggcctgcctcggcggacgacccgccttggatgcggtgaagaaggggctcccgcacgcgcctgcattgattaagcggaccaacagaaggattcccgagaggacatcacatcgagtggcagatcacgcgcaagtggtccgcgtgctcggattcttccagtgtcactcccaccccgcacaagcgttcgatgacgccatgactcaatttggtatgtcgagacacggactgctgcagctctttcgtagagtcggtgtcacagaactcgaggcccgctcgggcacactgcctcccgcctcccagcggtgggacaggattctccaagcgagcggtatgaaacgcgcgaagccttcacctacgtcaactcagacacctgaccaggcgagccttcatgcgttcgcagactcgctggagagggatttggacgcgccctcgcccatgcatgaaggggaccaaactcgcgcgtcagctagc

SEQ ID NO: 30 - DNA, 95 bp, Artificial Sequence
  Synthetic Construct
gacatctccaagtttgcagagaaagacaatatagttcttggagaaggtggaatcacactgagtggaggtcaacgagcaagaatttctttagcaag

SEQ ID NO: 31 - DNA, 177 bp, Artificial Sequence
  Synthetic Construct
ggatccagattgagcatactaaaagtgactctctaattttctatttttggtaataggacatctccaagtttgcagagaaagacaatatagttcttggagaaggtggaatcacactgagtggaggtcaacgagcaagaatttctttagcaaggtgaataactaattattggtggatcc

SEQ ID NO: 32 - DNA, 20 bp, Artificial Sequence
  Synthetic Construct
taatcatatatcacactatt

SEQ ID NO: 33 - protein, 1447 aa, Artificial Sequence
  Synthetic Construct
HMATVKFKYKGEEKEVDISKIKKVARVGKMISFTYDEGGGKTGEGAVSEKDAPKELLQMLEKQKKGGGGGGGGGGGCNTKKPNLDAELDQLLQGHYIKGYPKQYTYFLEDGKVKVSRPEGVKIIPPQSDRQKIVLQAHNLAHTGREATLLKIANLYWWPNMRKDVVKQLGRCQQCLITNASNKASGPILRPDRPQKPFDKFFIDYIGPLPPSQGYLYVLVVVDGMTGFTWLYPTKAPSTSATVKSLNVLTSIAIPKVIHSDQGAAFTSSTFAEWAKERGIHLEFSTPYHPQSSGKVERKNSDIKRLLTKLLVGRPTKWYDLLPVVQLALNNTYSPVLKYTPHQLLFGIKSNTPFANQDTLDLTREEELSLLQEIRTSLYHPSTPPASSRSWSPVVSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGRLTPEQVVAIASNGGGRPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRASAS

SEQ ID NO: 34 - DNA, 10101 bp, Artificial Sequence
  Synthetic Construct
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatccggatatagttcctcctttcagcaaaaaacccctcaagacccgtttagaggccccaaggggttatgctagttattgctcagcggtggcagcagccaactcagcttcctttcgggctttgttagcagccggatcctcagccaccgaccttccgcttcttctttggcccgctagctgacgcgcgagtttggtccccttcatgcatgggcgagggcgcgtccaaatccctctccagcgagtctgcgaacgcatgaaggctcgcctggtcaggtgtctgagttgacgtaggtgaaggcttcgcgcgtttcataccgctcgcttggagaatcctgtcccaccgctgggaggcgggaggcagtgtgcccgagcgggcctcgagttctgtgacaccgactctacgaaagagctgcagcagtccgtgtctcgacataccaaattgagtcatggcgtcatcgaacgcttgtgcggggtgggagtgacactggaagaatccgagcacgcggaccacttgcgcgtgatctgccactcgatgtgatgtcctctcgggaatccttctgttggtccgcttaatcaatgcaggcgcgtgcgggagccccttcttcaccgcatccaaggcgggtcgtccgccgaggcaggccagcgctacaagatgatcattagtgagtgcggccagcgcggggtccggcctcgaaagctgggccacgattgactccagtgcgggtctgcccccgccattggatgcaatagccactacctgctcaggcgtgagtcggccgtgcgcttggcacagcaccggcagcaatcgttgtacggtttccaacgcctgtttgccccctccattcgaggcaatggccacgacctgctctggggtcagtccgtgcgcttggcacagcacgggaagcaggcgctggacagtctcgagtgcctgtttcccgccaatgttggaggcgattgccacgacctgctctggggtcaggccgtgcgcttggcacagcaccggcagcaatcgttgtacggtttccaacgcctgtttgccccctccattcgaggcaatggccacgacctgctctggggtcagcccgtgcgcttggcacagcacaggcaacagcctctgtactgtttccaaggcttgctttcctccgtcgtggcttgcgatcgccacgacctgctctggggtcaatccgtgcgcttggcacagcacgggaagcaggcgctggacagtctcgagtgcctgtttcccgccaatgttggaggcgattgccacgacctgctctggggtcagcccgtgcgcttggcacagcacaggcaacagcctctgtactgtttccaaggcttgctttcctccgtcgtggcttgcgatcgccacgacctgctctggggtcaatccgtgcgcttggcacagcacgggaagcaggcgctggacagtctcgagtgcctgtttcccgccaatgttggaggcgattgccacgacctgctctggggtcagcccgtgcgcttggcacagcacaggcaacagcctctgtactgtttccaaggcttgctttcctccgtcgtggcttgcgatcgccacgacctgctctggggtcaagccgtgcgcttggcacagcaccggcagcaatcgttgtacggtttccaacgcctgtttgccccctccattcgaggcaatggccacgacctgctctggggtcagtccgtgcgcttggcacagcacgggaagcaggcgctggacagtctcgagtgcctgtttcccgccaatgttggaggcgattgccacgacctgctctggggtcaggccgtgcgcttggcacagcaccggcagcaatcgttgtacggtttccaacgcctgtttgccccctccattcgaggcaatggccacgacctgctctggggtcagtccgtgcgcttggcacagcacgggaagcaggcgctggacagtctcgagtgcctgtttcccgccaatgttggaggcgattgccacgacctgctctggggtcaggccgtgcgcttggcacagcaccggcagcaatcgttgtacggtttccaacgcctgtttgccccctccattcgaggcaatggccacgacctgctctggggtcagtccgtgcgcttggcacagcacgggaagcaggcgctggacagtctcgagtgcctgtttcccgccaatgttggaggcgattgccacgacctgctctggggtcagcccgtgcgcttggcacagcacaggcaacagcctctgtactgtttccaaggcttgctttcctccgtcgtggcttgcgatcgccacgacctgctctggggtcaagccgtgcgcttggcacagcaccggcagcaatcgttgtacggtttccaacgcctgtttgccccctccattcgaggcaatggccacgacctgctctggggtcagtccgtgcgcttggcacagcacgggaagcaggcgctggacagtctcgagtgcctgtttcccgccaatgttggaggcgattgccacgacctgctctggggtcagtccgtgcgcttggcacagcacgggaagcaggcgctggacagtctcgagtgcctgtttcccgccaatgttggaggcgattgccacgacctgctctggggtcaggttgaggggtgctcccgtgagcgcattgcgccacgcgtgcaccgcctcgaccgccgtgactcctccccgcttcgcgatcttcagcaactggcccgtgtccagctgaaggggaggccctctcagctctcccgcgaccgtcaacagggcctcaagcgctcgggctccgctccactgtttccccacaccgacgatcgcctcatgtgtggcttccggcaacgccgcaatcatgtcctggtacttgacggcgaccgtgccaagggctgcagggtgctgcgacaaggctacgatgtgggcatgtgtaaacccatgacccaccagcgcttcgtgatgctgcgcgactgtcgacctcactttgggcttgatcttttcctgctgctgctgtgaatatcccaaagttctcaaatctacttgcgctgcggggcttgcgtcggacggttgcgctgccctccgccttggtgcgggcttcgccctcggcggtcgggcagcggtgacggccacccgcatggttgggggtggtgcatccgcagctctcaatcccgactgcacctcatcccactccccggtggccgcctccgtatggtgcgccccaaacggaggaagggagtcgaacaacgatgtgttaaacagtgaggggtcgaactgcctaagcaggtctgagaacgagtcggccgaaaacgctgggctgggtgcgggtggggaagggagccgggtccgcgaaacaacaggagaccaggaacgagaggaggctggaggggtggatggatggtataaagaagtacgaatttcctgtaaaagagaaagttcttcttctctggtcaagtcaagtgtatcttgatttgcaaatggagtatttgattttataccaaataagagttgatgtggagtatattttaatacagggctataggtgttgtttaaagcaagttgtacaacaggcaataggtcataccactttgtgggtcttcctactagcagtttagttaaaagtcgttttatatcactatttttcctttccaccttaccactactttgggggtgataaggagtactgaattccaaatgtatacctctttcctttgcccattcagcaaaggttgaagaagtgaatgctgcaccttgatcagagtgaatcacctttggaattgcaatactagtgagtacattgagagatttaacagttgcgctagtagaaggagccttagtggggtataaccaagtgaatcctgtcattccatcaacaactactaatacatataggtatccctgtgaaggtggcaaaggtccaatatagtcaataaagaatttatcaaaaggtttttgaggcctatctggtcttagaataggaccagaggctttgttggaagcatttgtgattaaacactgttgacagcgtcctagttgtttaaccacatcctttctcatatttggccaccaataaaggttggcaatttttaaaagagtggcttcacgtccggtgtgagccaaattgtgggcttgaagcacaattttttgtctgtctgactgagggggaataattttaaccccttcaggtctggaaacttttactttgccatcttctaaaaaatatgtatattgtttgggatatccttttatataatgaccctgtaataattgatccaactctgcatccaggtttggttttttggtattacaaccaccgccaccgccaccgccaccgccaccgccttttttctgtttttccagcatctgcagcagttctttcggggcgtctttttcagaaaccgcgccttcacccgttttgccaccgccttcatcataggtaaagctaatcattttacccacacgcgcgacttttttgatttttgagatgtccacttctttttcttcgcctttgtatttgaatttaacggtcgccatatggggtccctgaaagaggacttcaaggctatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa